(19) United States
(12) Patent Application Publication
(10) Pub. No.: US 2026/0029324 A1
(43) Pub. Date: Jan. 29, 2026

(54) PROLIFERATION ASSAY FOR FIXED SOLID TUMORS

(71) Applicants: Ventana Medical Systems, Inc., Tucson, AZ (US); The Francis Crick Institute, London (GB); The Royal Marsden NHS Foundation Trust, London (GB)

(72) Inventors: Nelson ALEXANDER, Marana, AZ (US); Lisa GALLEGOS, Tucson, AZ (US); Brian Hanley, London (GB); Samra Turajlic, London (GB)

(21) Appl. No.: 19/344,940

(22) Filed: Sep. 30, 2025

Related U.S. Application Data
(63) Continuation of application No. PCT/US2024/022720, filed on Apr. 3, 2024.
(60) Provisional application No. 63/459,050, filed on Apr. 13, 2023.

(51) Int. Cl. G01N 15/1429; C12Q 1/37; G01N 1/30; G01N 15/10; G01N 15/14; G01N 21/47; G01N 21/64; G01N 33/68

(52) U.S. Cl. CPC G01N 15/1429; C12Q 1/37; G01N 1/30; G01N 21/47; G01N 21/6428; G01N 33/6872; G01N 33/6893; G01N 2015/1006; G01N 2015/1402; G01N 2021/6439; G01N 2333/4742

(57) ABSTRACT

Disclosed herein is a method of analyzing flow cytometry data for cells derived from homogenized whole tumor samples. In some embodiments, the present disclosure is directed to a cytometric assay for distinguishing between tumor cells expressing a cell proliferation marker and normal cells expressing the cell proliferation marker. In some embodiments, the present disclosure is also directed to quantifying a percentage of normal cells expressing a cell proliferation marker and a percentage of tumor cells expressing the cell proliferation marker.

### CROSS REFERENCE TO RELATED APPLICATIONS

The present application is a continuation of International Application No. PCT/US2024/022720 filed on Apr. 3, 2024, which application claims the benefit of the filing date of U.S. Provisional Patent Application No. 63/459,050, filed on Apr. 13, 2023, the disclosures of which are hereby incorporated by reference herein in their entireties.

### FIELD OF THE INVENTION

The present disclosure is directed to cytometry assays, such as cytometry assays for distinguishing between tumor cells expressing a cell proliferation marker and normal cells expressing the cell proliferation marker.

### BACKGROUND OF THE DISCLOSURE

Cancer is a disease marked by the uncontrolled proliferation of abnormal cells. In normal tissue, cells divide and organize within the tissue in response to signals from surrounding cells, resulting in normal cellular behavior that is carefully orchestrated by the tissue context. Cancer cells do not respond to growth-limiting contextual cues from the surrounding tissue, and they often harbor genetic alterations that drive them to proliferate and form a tumor. As the growth of a tumor progresses, genetic and phenotypic alterations continue to accumulate, allowing populations of cancer cells to overcome additional “checkpoints,” such as an anti-tumor immune response, and manifesting as a more aggressive growth phenotype of the cancer cells. If left untreated, metastasis, the spread of cancer cells to distant areas of the body by way of the lymphatic system or bloodstream, may ensue. Metastasis results in the formation of secondary tumors at multiple sites, damaging healthy tissue. Most cancer death is caused by such secondary tumors. Timely diagnosis and treatment of cancer enhances the likelihood of a successful outcome.

Breast cancer is the most common cancer diagnosed in women in the United States. Unfortunately, breast cancer recurrences will occur, with risks tied strongly to the original TN status (from 10 to 41%, depending on tumor diameter and nodal status). (Pan et. al., “20-Year Risks of Breast-Cancer Recurrence after Stopping Endocrine Therapy at 5 Years.” N Engl J Med 2017; 377(19): 1836-1846). This observation data suggests that clinically occult tumor (“micrometastasis”) will be present in a fraction of cases after surgery.

“Primary surgery for breast cancer is accomplished by lumpectomy followed by whole-breast irradiation or by mastectomy.” (Chew H K. “Adjuvant therapy for breast cancer: who should get what?” West J Med. 2001 April;174(4):284-7). Adjuvant therapies are additional treatments that help kill any cancer cells remaining in the body after a primary therapy, such as one or more surgical interventions. Adjuvant treatment for breast cancer may include chemotherapy, systemic therapy with cytotoxic chemotherapy, radiation, hormone therapy (e.g., endocrine therapy), biological therapy, and targeted therapies.

Neoadjuvant chemotherapy is a course of cancer treatment used prior to surgery. It is believed that this treatment helps shrink cancerous tumors to make them easier to remove. Neoadjuvant chemotherapy may also kill cancerous tissue that is not yet visible on imaging tests. Neoadjuvant endocrine therapy is another course of cancer treatment used prior to surgery. It is similarly believed that this treatment helps shrink tumors to make them easier to remove. Additionally, biomarkers such as Ki-67 can be measured before and after neoadjuvant endocrine therapy to determine if a more aggressive treatment (e.g., chemotherapy) is required in the adjuvant setting. (See Smith et. Al., “Long-term Outcome and Prognostic Value of Ki-67 After Perioperative Endocrine Therapy in Postmenopausal Women with Hormone-Sensitive Early Breast Cancer (POETIC): an Open-Label, Multicentre, Parallel-Group, Randomised, Phase 3 Trial,” Lancet Oncol 2020; 21:1443-54).

The current diagnostic test that determines whether breast cancer patients receive adjuvant chemotherapy is OncotypeDx (developed by Genomic Health, Inc., USA), a 21 gene panel that measures 16 cancer genes (remainder are reference genes), grouped into categories: proliferation, subtype, invasion, and other. (See Mi Jeong Kwon, “Emerging immune Gene Signatures as Prognostic or Predictive Biomarkers in Breast Cancer,” Arch. Pharm. Res. (2019) 42:947-961). The predictive value of each group of genes determines a weighting scheme for the score, with the proliferation group overwhelmingly dominating the score, followed by subtype, and the remainder of the genes contribute very minimally. As such, it has been noted that the proliferation phenotype of breast tumors must be critical for determining whether patients receive adjuvant chemotherapy. Tumors, however, are composed of many different types of cells, and the OncotypeDx readout will not distinguish between tumor cell proliferation and other cell types in the tumor proliferating, such as immune cells. Distinguishing between these different proliferation phenotypes may provide more granular diagnostic information than the Oncotype Dx diagnostic test in use today. In addition, the Oncotype Dx test has a two-week turnaround time, and any test with a shorter turnaround time would be an improvement.

### BRIEF SUMMARY OF THE DISCLOSURE

Applicant has developed a cytometric assay that distinguishes between tumor and normal cell proliferation in single cells dissociated from a tumor sample. In particular, the assay of the present disclosure distinguishes between tumor cell proliferation (adding to the risk of recurrence) versus immune cell proliferation (potentially leading to a lower risk of recurrence). This, it is believed, allows for a more granular and specific proliferation readout, which is important since proliferation is the strongest factor tied to risk of recurrence. Additionally, the cytometric assay of the present disclosure is quick, providing results within one day.

A first aspect of the present disclosure is a method of quantifying a percentage of normal cells and a percentage of tumor cells expressing a cell proliferation marker comprising: obtaining a sample; staining cells within a first aliquot derived from the sample for the presence of at least one cell proliferation marker; staining cells within a second aliquot derived from the sample for the presence of at least one tumor marker; optionally counterstaining cells within the first and second aliquots for the presence of DNA; obtaining cytometric data for the stained cells within each of the first and second aliquots; based on the obtained cytometric data, quantifying the percentage of the normal cells and the percentage of the tumor cells in each of a cell proliferation marker positive tumor cell population and a cell proliferation marker positive normal cell population. In some embodiments, the cytometric data is derived from flow cytometry. In some embodiments, the cytometric data comprises one or more scatter plots. In some embodiments, the cytometric data comprises scatter plots of fluorescence versus side scatter for the stained cells within each of the first and second aliquots. In some embodiments, the stained cells are gated into at least one of the cell proliferation marker positive tumor cell population or the cell proliferation marker positive normal cell population.

In some embodiments, the at least one tumor marker is an epithelial marker. In some embodiments, the epithelial marker is a cytokeratin. In some embodiments, the cytokeratin is selected from high molecular weight cytokeratins and/or low molecular weight cytokeratins. In some embodiments, the cytokeratin is selected from CK8/18, or a pan-cytokeratin marker recognizing cytokeratins 1-8, 10, 14-16 and 19.

In some embodiments, the at least one cell proliferation marker is selected from the group consisting of Ki-67, Ki-S5, Ki-S2, p21, p27, Caspases, BAD, CD95, fas-ligand, parp-proteins. In some embodiments, the at least one cell proliferation biomarker is Ki-67. In some embodiments, the at least one tumor marker is a cytokeratin and the at least one cell proliferation marker is Ki-67. In some embodiments, the at least one tumor marker is CK8/18 and the at least one cell proliferation marker is Ki-67.

In some embodiments, the gating of the stained cells comprises performing at least two gatings. In some embodiments, a first gating of the at least two gatings is performed to identify cells that are positive for the tumor cell marker. In some embodiments, the first gating comprises: (i) obtaining a scatter plot of fluorescence versus side scatter for a negative control aliquot derived from the sample; (ii) positioning a vertical quadrant gate such that fewer than a predetermined percentage of the stained cells in the negative control aliquot scatter plot are located to the right of the vertical quadrant gate; and (iii) positioning a horizontal quadrant gate in the generated scatter plot for the second aliquot such that fewer than a predetermined percentage of the stained cells in the generated scatter plot for the second aliquot are located in a lower right corner of the generated scatter plot of the second aliquot. In some embodiments, the negative control aliquot is incubated with one or more detection reagents. In some embodiments, a second gating comprises mapping the first gating to the generated scatter plot corresponding to the first aliquot.

In some embodiments, the method further comprises optionally assessing DNA content within at least the first and second aliquots, such as to confirm the at least two gatings.

In some embodiments, the method further comprises staining cells within a third aliquot derived from the sample for the presence of at least one normal cell marker.

In some embodiments, the obtained sample is derived from a heterogenous input sample which has been mechanically and/or chemically dissociated, and wherein the sample comprises substantially uniformly distributed cells. In some embodiments, the obtained sample is derived from a heterogenous input sample which has been mechanically dissociated (without further chemical dissociation).

In some embodiments, the heterogeneous input sample is derived from one or more surgical resections and/or residual surgical material. In some embodiments, the residual surgical tumor sample is a specimen that would have otherwise been destroyed. In some embodiments, an entire obtained residual surgical tumor sample is fixed in an aldehyde-based fixative but not paraffin embedded, and wherein all the fixed but not paraffin embedded residual surgical sample is homogenized. In some embodiments, an entire obtained residual surgical tumor sample is fixed, such as in an aldehyde-based fixative, and wherein all the fixed residual surgical sample is homogenized. In some embodiments, the residual surgical tumor sample is not derived from tissue specimens used for diagnostic purposes. In some embodiments, the residual surgical tumor sample is not derived from tissue specimens used TNM staging.

In some embodiments, a ratio of cells in any aliquot derived from the sample is substantially similar to the ratio of cells in the heterogeneous input sample. In some embodiments, the heterogeneous input sample is at least 1 cm in diameter at its widest point. In some embodiments, the heterogeneous input sample is at least 2 cm in diameter at its widest point. In some embodiments, the heterogeneous input sample is at least 5 cm in diameter at its widest point. In some embodiments, the heterogeneous input sample has a volume of at least about 10 cm³.

In some embodiments, the obtained sample comprises dissociated cells. In some embodiments, the obtained sample comprises a homogenized fixed tissue sample, such as a tissue sample fixed in an aldehyde-based fixative.

In some embodiments, the method further comprises sequencing genomic material isolated from cells within the cell proliferation marker positive tumor cell population. In some embodiments, the sequencing comprises next generation sequencing. In some embodiments, the sequencing comprises single cell sequencing. In some embodiments, the sequencing comprises single nucleus sequencing. In some embodiments, the sequencing comprises long-read sequencing.

In some embodiments, the staining of the first aliquot of the sample comprises contacting the first aliquot with a primary antibody specific for the cell proliferation marker (e.g., an anti-Ki-67 antibody specific to Ki-67) to form a cell proliferation marker-primary antibody complex. In some embodiments, the primary antibody specific for the cell proliferation antibody is an anti-Ki-67 monoclonal antibody. In some embodiments, the method further comprises contacting the first aliquot of the sample with a secondary antibody specific for the primary antibody specific for the cell proliferation marker. In some embodiments, the secondary antibody is conjugated to a first fluorescent label.

In some embodiments, the staining of the second aliquot of the sample comprises contacting the second aliquot with a primary antibody specific for the tumor marker to form a tumor marker-primary antibody complex. In some embodiments, the method further comprises contacting the second aliquot of the sample with a secondary antibody specific for the primary antibody specific for the tumor marker. In some embodiments, the secondary antibody specific for the primary antibody specific for the tumor marker is conjugated to a second fluorescent label.

A second aspect of the present disclosure is a method of assessing a percentage of cell proliferation marker positive normal cells and a percentage of cell proliferation marker positive tumor cells comprising: obtaining at least two aliquots of a sample, wherein cells within a first aliquot of the at least two aliquots of the sample are fluorescently stained for the presence of a cell proliferation marker, and wherein cells within a second aliquot of the at least two aliquots of the sample are fluorescently stained for the presence of a tumor marker; generating a first scatter plot of fluorescence versus side scatter for the fluorescently stained cells within the first aliquot of the sample; generating a second scatter plot of fluorescence versus side scatter for the fluorescently stained cells within the second aliquot of the sample; and performing at least two gating operations, such as two sequential gating operations, using at least the first and second generated scatter plots to assess the percentage of cell proliferation positive normal cells and the percentage of cell proliferation positive tumor cells.

In some embodiments, the method further comprises counterstaining cells within the first and second aliquots for the presence of DNA. In some embodiments, the method further comprises obtaining a third aliquot of the sample, wherein cells within the third aliquot of the sample are fluorescently stained for the presence of a normal cell marker. In some embodiments, the tumor marker is an epithelial marker. In some embodiments, the epithelial marker is a cytokeratin. In some embodiments, the cytokeratin is selected from CK1, CK2, CK3, CK4, CK5, CK6, CK7, CK8, and CK9. In some embodiments, the cytokeratin is selected from CK10, CK12, CK13, CK14, CK16, CK17, CK18, CK19, and CK20. In some embodiments, the cytokeratin is selected from the group consisting of CK8/18, or a pan-cytokeratin marker recognizing cytokeratins 1-8, 10, 14-16 and 19. In some embodiments, the cell proliferation biomarker is selected from the group consisting of Ki-67, Ki-S5, Ki-S2, p21, p27, Caspases, BAD, CD95, fas-ligand, parp-proteins. In some embodiments, the cell proliferation biomarker is Ki-67. In some embodiments, the at least one tumor marker is a cytokeratin and the at least one cell proliferation marker is Ki-67. In some embodiments, the at least one tumor marker is CK8/18 and the at least one cell proliferation marker is Ki-67.

In some embodiments, the method further comprises obtaining a negative control aliquot, wherein cells within the negative control aliquot are incubated with one or more detection reagents; and wherein cytometry data is generated for the negative control aliquot.

In some embodiments, the method further comprises generating a negative control scatter plot of fluorescence versus side scatter for the cells within the negative control aliquot.

In some embodiments, a first gating of the at least two gatings is performed to identify cells that are positive for the tumor cell marker. In some embodiments, a second gating of the at least two gatings is performed to identify cell proliferation marker positive normal cells and cell proliferation marker positive tumor cells. In some embodiments, the obtained sample is a representative sample derived from a heterogenous input sample which has been mechanically and/or chemically dissociated, and wherein the representative sample comprises substantially uniformly distributed cells.

In some embodiments, the heterogeneous input sample is derived from one or more surgical resections and/or residual surgical material. In some embodiments, the residual surgical tumor sample is a specimen that would have otherwise been destroyed. In some embodiments, an entire obtained residual surgical tumor sample is fixed in an aldehyde-based fixative but not paraffin embedded, and wherein all the fixed but not paraffin embedded residual surgical sample is homogenized. In some embodiments, an entire obtained residual surgical tumor sample is fixed in an aldehyde-based fixative, and wherein all the fixed residual surgical sample is homogenized. In some embodiments, the residual surgical tumor sample is not derived from tissue specimens used for diagnostic purposes. In some embodiments, the residual surgical tumor sample is not derived from tissue specimens used TNM staging.

In some embodiments, a ratio of cells in any aliquot derived from the representative sample is substantially similar to the ratio of cells in the heterogeneous input sample. In some embodiments, the heterogeneous input sample has a volume of at least about 10 cm³. In some embodiments, the obtained sample comprises dissociated cells. In some embodiments, the obtained sample comprises a homogenized fixed tissue sample.

In some embodiments, the method further comprises sorting the cell proliferation marker positive cells into a cell proliferation marker positive normal cell population and a cell proliferation marker positive tumor cell population. In some embodiments, the method further comprises sequencing genomic material isolated from cells within the cell proliferation marker positive tumor cell population. In some embodiments, the sequencing comprises next generation sequencing. In some embodiments, the sequencing comprises single cell sequencing. In some embodiments, the sequencing comprises single nucleus sequencing. In some embodiments, the sequencing comprises long read sequencing.

A third aspect of the present disclosure is a method of assessing a percentage of cell proliferation marker positive normal cells and a percentage of cell proliferation marker positive tumor cells comprising: obtaining a residual surgical tumor sample from a human subject, wherein the residual surgical tumor material is fixed but not embedded within paraffin, and wherein the residual surgical tumor material has not been deparaffinized; mechanically blending the obtained residual surgical tumor material to provide a representative sample, wherein any subpopulations of cells that were originally spatially segregated within the residual surgical tumor material are homogeneously distributed throughout the representative sample, and wherein any aliquot removed from the representative sample comprises one or more populations of subclones at a proportion at which they existed within the obtained residual surgical tumor sample; obtaining at least two aliquots of the representative sample, wherein cells within a first aliquot of the at least two aliquots of the representative sample are fluorescently stained for the presence of a cell proliferation marker, and wherein cells within a second aliquot of the at least two aliquots of the representative sample are fluorescently stained for the presence of a tumor marker; generating a first scatter plot of fluorescence versus side scatter for the fluorescently stained cells within the first aliquot of the representative sample; generating a second scatter plot of fluorescence versus side scatter for the fluorescently stained cells within the second aliquot of the representative sample; and performing at least two gating operations, such as two sequential gating operations, using at least the first and second generated scatter plots to assess the percentage of cell proliferation positive normal cells and the percentage of cell proliferation positive tumor cells.

In some embodiments, the method further comprises counterstaining cells within the first and second aliquots for the presence of DNA. In some embodiments, the method further comprises obtaining a third aliquot of the sample, wherein cells within the third aliquot of the sample are fluorescently stained for the presence of a normal cell marker. In some embodiments, the tumor marker is an epithelial marker. In some embodiments, the epithelial marker is a cytokeratin. In some embodiments, the cytokeratin is selected from CK1, CK2, CK3, CK4, CK5, CK6, CK7, CK8, and CK9. In some embodiments, the cytokeratin is selected from CK10, CK12, CK13, CK14, CK16, CK17, CK18, CK19, and CK20. In some embodiments, the cytokeratin is selected from the group consisting of CK8/18, or a pan-cytokeratin marker recognizing cytokeratins 1-8, 10, 14-16 and 19. In some embodiments, the cell proliferation biomarker is selected from the group consisting of Ki-67, Ki-S5, Ki-S2, p21, p27, Caspases, BAD, CD95, fas-ligand, parp-proteins. In some embodiments, the cell proliferation biomarker is Ki-67. In some embodiments, the at least one tumor marker is a cytokeratin and the at least one cell proliferation marker is Ki-67. In some embodiments, the at least one tumor marker is CK8/18 and the at least one cell proliferation marker is Ki-67.

In some embodiments, the method further comprises obtaining a negative control aliquot, wherein cells within the negative control aliquot are incubated with one or more detection reagents; and wherein cytometry data is generated for the negative control aliquot.

In some embodiments, the method further comprises generating a negative control scatter plot of fluorescence versus side scatter for the cells within the negative control aliquot.

In some embodiments, a first gating of the at least two gatings is performed to identify cells that are positive for the tumor cell marker. In some embodiments, a second gating of the at least two gatings is performed to identify cell proliferation marker positive normal cells and cell proliferation marker positive tumor cells. In some embodiments, the obtained sample is a representative sample derived from a heterogenous input sample which has been mechanically and/or chemically dissociated, and wherein the representative sample comprises substantially uniformly distributed cells.

In some embodiments, the heterogeneous input sample is derived from one or more surgical resections and/or residual surgical material. In some embodiments, the residual surgical tumor sample is a specimen that would have otherwise been destroyed. In some embodiments, an entire obtained residual surgical tumor sample is fixed in an aldehyde-based fixative but not paraffin embedded, and wherein all the fixed but not paraffin embedded residual surgical sample is homogenized. In some embodiments, an entire obtained residual surgical tumor sample is fixed in an aldehyde-based fixative, and wherein all the fixed residual surgical sample is homogenized. In some embodiments, the residual surgical tumor sample is not derived from tissue specimens used for diagnostic purposes. In some embodiments, the residual surgical tumor sample is not derived from tissue specimens used TNM staging.

In some embodiments, a ratio of cells in any aliquot derived from the representative sample is substantially similar to the ratio of cells in the heterogeneous input sample. In some embodiments, the heterogeneous input sample has a volume of at least about 10 cm³. In some embodiments, the obtained sample comprises dissociated cells. In some embodiments, the obtained sample comprises a homogenized fixed tissue sample.

In some embodiments, the method further comprises sorting the cell proliferation marker positive cells into a cell proliferation marker positive normal cell population and a cell proliferation marker positive tumor cell population. In some embodiments, the method further comprises sequencing genomic material isolated from cells within the cell proliferation marker positive tumor cell population. In some embodiments, the sequencing comprises next generation sequencing. In some embodiments, the sequencing comprises single cell sequencing. In some embodiments, the sequencing comprises single nucleus sequencing. In some embodiments, the sequencing comprises long read sequencing.

In some embodiments, the method further comprises processing another portion of the representative sample to generate at least one disassociated cell, wherein the at least one disassociated cell is a normal cell, a cancer cell, or a bacterial cell, and wherein the at least one disassociated cell is disposed on or affixed to at least one glass slide. In some embodiments, the at least one disassociated cell disposed on or affixed to at least one glass slide is subjected to histological analysis, wherein the histological analysis is hematoxylin and eosin (“H&E”) staining, immunohistochemical (“IHC”) staining, in situ hybridization (“ISH”) staining, or fluorescent in situ hybridization (“FISH”) staining.

A fourth aspect of the present disclosure is a method of quantifying a percentage of normal cells and a percentage of tumor cells expressing a cell proliferation marker comprising: obtaining a residual surgical tumor sample from a human subject, wherein the residual surgical tumor material is fixed but not embedded within paraffin, and wherein the residual surgical tumor material has not been deparaffinized; mechanically blending the obtained residual surgical tumor material to provide representative sample, wherein any subpopulations of cells that were originally spatially segregated within the residual surgical tumor material are homogeneously distributed throughout the representative sample, and wherein any aliquot removed from the representative sample comprises one or more populations of subclones at a proportion at which they existed within the obtained residual surgical tumor sample; staining cells within a first aliquot derived from the representative sample for the presence of at least one cell proliferation marker; staining cells within a second aliquot derived from the representative sample for the presence of at least one tumor marker; optionally counterstaining cells within the first and second aliquots for the presence of DNA; generating scatter plots of fluorescence versus side scatter for the stained cells within each of the first and second aliquots; based on the obtained scatter plots, gating the stained cells into at least one of a cell proliferation marker positive tumor cell population and a cell proliferation marker positive normal cell population; and quantifying the percentage of the normal cells and the percentage of the tumor cells in each of the cell proliferation marker positive tumor cell and cell proliferation marker positive normal cell populations.

In some embodiments, the at least one tumor marker is an epithelial marker. In some embodiments, the epithelial marker is a cytokeratin. In some embodiments, the cytokeratin is selected from high molecular weight cytokeratins and/or low molecular weight cytokeratins. In some embodiments, the cytokeratin is selected from CK8/18, or a pan-cytokeratin marker recognizing cytokeratins 1-8, 10, 14-16 and 19.

In some embodiments, the at least one cell proliferation biomarker is selected from the group consisting of Ki-67, Ki-S5, Ki-S2, p21, p27, Caspases, BAD, CD95, fas-ligand, parp-proteins. In some embodiments, the at least one cell proliferation biomarker is Ki-67. In some embodiments, the at least one tumor marker is a cytokeratin and the at least one cell proliferation marker is Ki-67. In some embodiments, the at least one tumor marker is CK8/18 and the at least one cell proliferation marker is Ki-67.

In some embodiments, the gating of the stained cells comprises performing at least two gatings, such as two sequential gatings. In some embodiments, a first gating of the at least two gatings is performed to identify cells that are positive for the tumor cell marker. In some embodiments, the first gating comprises: (i) obtaining a scatter plot of fluorescence versus side scatter for a negative control aliquot derived from the sample; (ii) positioning a vertical quadrant gate such that fewer than a predetermined percentage of the stained cells in the negative control aliquot scatter plot are located to the right of the vertical quadrant gate; and (iii) positioning a horizontal quadrant gate in the generated scatter plot for the second aliquot such that fewer than a predetermined percentage of the stained cells in the generated scatter plot for the second aliquot are located in a lower right corner of the generated scatter plot of the second aliquot. In some embodiments, the negative control aliquot is incubated with one or more detection reagents. In some embodiments, a second gating comprises mapping the first gating to the generated scatter plot corresponding to the first aliquot.

In some embodiments, the method further comprises optionally assessing DNA content within at least the first and second aliquots to confirm the at least two gatings.

In some embodiments, the method further comprises staining cells within a third aliquot derived from the sample for the presence of at least one normal cell marker.

In some embodiments, the obtained sample is derived from a heterogenous input sample which has been mechanically and/or chemically dissociated, and wherein the sample comprises substantially uniformly distributed cells. In some embodiments, the obtained sample is derived from a heterogenous input sample which has been mechanically dissociated (without further chemical dissociation).

In some embodiments, the heterogeneous input sample is derived from one or more surgical resections and/or residual surgical material. In some embodiments, the residual surgical tumor sample is a specimen that would have otherwise been destroyed. In some embodiments, an entire obtained residual surgical tumor sample is fixed, such as fixed in an aldehyde-based fixative but not paraffin embedded, and wherein all the fixed but not paraffin embedded residual surgical sample is homogenized. In some embodiments, an entire obtained residual surgical tumor sample is fixed in an aldehyde-based fixative, and wherein all the fixed residual surgical sample is homogenized. In some embodiments, the residual surgical tumor sample is not derived from tissue specimens used for diagnostic purposes. In some embodiments, the residual surgical tumor sample is not derived from tissue specimens used TNM staging.

In some embodiments, a ratio of cells in any aliquot derived from the sample is substantially similar to the ratio of cells in the heterogeneous input sample. In some embodiments, the heterogeneous input sample is at least 1 cm in diameter at its widest point. In some embodiments, the heterogeneous input sample is at least 2 cm in diameter at its widest point. In some embodiments, the heterogeneous input sample is at least 5 cm in diameter at its widest point. In some embodiments, the heterogeneous input sample has a volume of at least about 10 cm³.

In some embodiments, the obtained sample comprises dissociated cells. In some embodiments, the obtained sample comprises a homogenized fixed tissue sample.

In some embodiments, the method further comprises sequencing genomic material isolated from cells within the cell proliferation marker positive tumor cell population. In some embodiments, the sequencing comprises next generation sequencing. In some embodiments, the sequencing comprises single cell sequencing. In some embodiments, the sequencing comprises single nucleus sequencing. In some embodiments, the sequencing comprises long-read sequencing.

In some embodiments, the staining of the first aliquot of the sample comprises contacting the first aliquot with a primary antibody specific for the cell proliferation marker to form a cell proliferation marker-primary antibody complex. In some embodiments, the primary antibody specific for the cell proliferation antibody is an anti-Ki-67 monoclonal antibody. In some embodiments, the method further comprises contacting the first aliquot of the sample with a secondary antibody specific for the primary antibody specific for the cell proliferation marker. In some embodiments, the secondary antibody is conjugated to a first fluorescent label.

In some embodiments, the staining of the second aliquot of the sample comprises contacting the second aliquot with a primary antibody specific for the tumor marker to form a tumor marker-primary antibody complex. In some embodiments, the method further comprises contacting the second aliquot of the sample with a secondary antibody specific for the primary antibody specific for the tumor marker. In some embodiments, the secondary antibody specific for the primary antibody specific for the tumor marker is conjugated to a second fluorescent label.

### BRIEF DESCRIPTION OF THE FIGURES

For a general understanding of the features of the disclosure, reference is made to the drawings. In the drawings, like reference numerals have been used throughout to identify identical elements.

FIG. 1A sets forth a flow chart illustrating the steps of obtaining and assessing flow cytometry data derived from a representative sample in accordance with one embodiment of the present disclosure.

FIG. 1B sets forth a flow chart illustrating the steps of obtaining and assessing flow cytometry data derived from a representative sample in accordance with one embodiment of the present disclosure.

FIG. 1C sets forth a flow chart illustrating the steps of obtaining and assessing flow cytometry data derived from a representative sample in accordance with one embodiment of the present disclosure.

FIG. 2 sets forth a general procedure for obtaining dissociated cells, staining of one or more antigens on the surface or within the dissociated cells, sort the stained dissociated cells (physically or digitally), and analyzing one or more cell populations of interest based on acquired flow cytometry fluorescence data.

FIG. 3 illustrates provides an overview of a method of generating a representative sample in accordance with one embodiment of the present disclosure.

FIG. 4A illustrates a method of preparing a first stained aliquot derived from a representative sample, the first stained aliquot being stained for the presence of one or more cell proliferation markers (e.g., Ki-67) and optionally counterstained (e.g., DAPI).

FIG. 4B illustrates a method of preparing a second stained aliquot derived from a representative sample, the second stained aliquot being stained for the presence of one or more tumor markers (e.g., a cytokeratin marker) and optionally counterstained (e.g., DAPI).

FIG. 4C illustrates a method of preparing a third stained aliquot derived from a representative sample, the third stained aliquot being incubated with one or more detection reagents (such as fluorescent detection reagents used to label the cells within the first and second aliquots) and optionally counterstained (e.g., DAPI).

FIG. 5 sets forth a method of performing a sequential gating to identify cell proliferation marker positive normal cells and cell proliferation marker positive tumor cells in a sample in accordance with one embodiment of the present disclosure.

FIGS. 6A and 6B illustrate a method of performing a first gating in accordance with one embodiment of the present disclosure.

FIG. 7A provides two scatter plots derived from flow cytometry data. Each scatter plot plots fluorescence intensity on the x-axis and side scatter content on the y-axis. The left-most panel illustrates a scatter plot derived from flow cytometry analysis of a negative control aliquot, i.e., an aliquot derived from a representative sample that is not stained for the presence of a specific biomarker, but rather is incubated with one or more detection reagents and/or counterstained. The right-most scatter plot is derived from flow cytometry analysis of a tumor marker aliquot, i.e., an aliquot derived from a representative sample that is stained for at least the presence of a tumor marker, such as a cytokeratin marker.

FIG. 7B provides two histograms of DNA content. Panel 1 illustrates the DNA content from all cells; while Panel 2 illustrates the DNA content of cells staining positive for a tumor maker. The histograms of FIG. 7B may, in some embodiments, be used to confirm a gating operation, such as a first gating operation.

FIG. 8 illustrates a method of performing a second gating in accordance with one embodiment of the present disclosure.

FIG. 9A provides a scatter plot derived from flow cytometry data. The scatter plot plots fluorescence intensity on the x-axis and side scatter content on the y-axis. The illustrated scatter plot is derived from flow cytometry analysis of a cell proliferation marker aliquot.

FIG. 9B provides three histograms of DNA content. Panel 1 illustrates the DNA content from all cell proliferation marker positive cells; while Panel 2 illustrates the DNA content of cell proliferation marker positive normal cells; and Panel 3 illustrates the DNA content of cell proliferation marker positive tumor cells. The histograms of FIG. 9B may, in some embodiments, be used to confirm a gating operation, such as a second gating operation.

FIGS. 10A-10D provide an example of linked gating quadrants across plots for the prepared negative control, normal marker (CD3), tumor marker (CK), and proliferation marker aliquots. Placement of the horizontal quadrant marker is optimized such that normal marker (CD3), and tumor marker (CK) populations are maximized, and DNA analysis is used to either guide placement of the gates or to confirm the accuracy of the placement of the gates. With this analysis, DNA content from high side scatter (HSSC) Ki-67+ cells appear aneuploid like the CK+ population; and DNA content from low side scatter (LSSC) Ki-67+ cells appear diploid like the CD3+ population. FIG. 10A shows a strategy of linking gating quadrants across scatter plots to analyze cells dissociated from tumor tissue. Negative control, normal marker aliquot (CD3 stained cells), and tumor marker aliquot (CK stained cells) are shown from left to right. In each graph, side scatter is plotted on the vertical axis, and marker intensity is on the horizontal axis. In some embodiments, a vertical quadrant gate is placed at the right edge of the negative control cells. In some embodiments, a horizontal marker is placed so that most CD3 positive cells are in the lower right quadrant, and most CK positive cells are in the upper right quadrant. FIG. 10B depicts the DNA analysis of the populations in A using DAPI staining. DNA histograms from all cells, CD3+ cells, and CK+ cells are shown from left to right. Note the presence of two peaks of DNA staining in the DNA from all cells, a diploid peak and aneuploid peak. In CD3+ cells, only the diploid peak is observed, and in CK+ cells only the aneuploid peak is observed. FIG. 10C illustrates the gating of Ki-67 positive cells. The gates established for CD3 and CK are mapped onto the graph of Ki-67 stained cells. FIG. 10D illustrates that HSSC Ki-67+ cells are presumed to be proliferating tumor cells, while LSSC Ki-67+ cells are presumed to be proliferating normal cells. DNA content from HSSC Ki-67+ cells appear aneuploid like the CK+ population, and DNA content from LSSC Ki-67+ cells appear diploid like the CD3+ population, confirming the best placement of the gates.

FIGS. 11A-11D provide an example of linked gating quadrants across plots for the prepared negative control, normal marker (CD3), tumor marker (CK), and proliferation marker aliquots. Placement of the horizontal quadrant marker is optimized such that normal marker (CD3), and tumor marker (CK) populations are maximized, and DNA analysis is used to either guide placement of the gates or to confirm the accuracy of the placement of the gates. With this analysis, DNA content from HSSC Ki-67+ cells appear aneuploid like the CK+ population, and DNA content from LSSC Ki-67+ cells appear diploid like the CD3+ population. FIG. 11A shows a strategy of linking gating quadrants across scatter plots to analyze cells dissociated from tumor tissue. Negative control, normal marker aliquot (CD3 stained cells), and tumor marker aliquot (CK stained cells) are shown from left to right. In each graph, side scatter is plotted on the vertical axis, and marker intensity is on the horizontal axis. In some embodiments, a vertical quadrant gate is placed at the right edge of the negative control cells. In some embodiments, a horizontal marker is placed so that most CD3 positive cells are in the lower right quadrant, and most CK positive cells are in the upper right quadrant. FIG. 11B depicts DNA analysis of the populations in A using DAPI staining. DNA histograms from all cells, CD3+ cells, and CK+ cells are shown from left to right. Note that in diploid tumors, all cells are diploid, so it is not possible to use DNA content to confirm the gating strategy. FIG. 11C illustrating the gating of Ki-67 positive cells. The gates established for CD3 and CK are mapped onto the graph of Ki-67 stained cells. FIG. 11D illustrates that HSSC Ki-67+ cells are presumed to be proliferating tumor cells, and LSSC Ki-67+ cells are presumed to be proliferating normal cells. Although ploidy cannot be used to confirm placement of gates, DNA from HSSC Ki-67+ cells have an enhanced G2 DNA content, consistent with the identification of these cells as a proliferating population.

FIGS. 12A and 12B providing an example showing a case with evidence of high side scatter Ki-67 not coming from tumor population, i.e., the CK+ cells are aneuploid and the HSSC Ki-67+ cells are diploid. These cases should be excluded from analysis. Only 3 out of 48 cases shows such an example, with consistently low overall Ki-67 staining. FIG. 12A depicts dissociated cells from fixed tumor tissue. From left to right, these consist of negative control cells, cells stained for cytokeratin (CK) and cells stained for Ki-67. The gating strategy was performed in accordance with the methods described herein. FIG. 12B illustrates DNA content from all cells, CK+ cells, and HSSC Ki-67+ cells. Note that in this example, DNA from HSSC Ki-67+ cells are diploid, while the tumor consists of aneuploid cells. Examples such as these should be excluded from analysis since the proliferating HSSC cells are not tumor cells. In a group of 51 cases, 3 such cases were identified, all with very low percentage of HSSC Ki-67+ cells (less than 1%).

FIGS. 13A-13B provide a summary of the testing of tumor and normal cell proliferation across 48 breast cancer cases. FIG. 13A provides boxplots showing the distribution of CD3 positive and Cytokeratin (CK) positive percentages across a dataset of 48 breast cancer cases. Each data point represents an individual case, and data points were jittered to avoid overplotting. FIG. 13B depicts an example of a scatter plot of side scatter vs. marker staining using a gating strategy defined in previous figures. These data are used to determine the percentages plotted in FIG. 13A. Note that “CD3” refers to the refers to the lower right quadrant of the top graph and “CK” refers to the upper right quadrant of the bottom graph for each case.

FIGS. 14A-14B provide a summary of the testing of tumor and normal cell proliferation across 48 breast cancer cases. FIG. 14A provides boxplots showing the distribution of total Ki-67 (Ki-67ALL), high side scatter Ki-67 (Ki-67 HSSC) and low side scatter Ki-67 (Ki-67 LSSC) percentages across a dataset of 48 breast cancer cases. Each data point represents an individual case, and data points were jittered to avoid overplotting. FIG. 14B depicts an example of a scatter plot of side scatter and Ki-67 marker staining using a gating strategy defined in previous figures. These data are used to determine the percentages plotted in FIG. 14A. Note that “Ki-67 ALL” refers to the total percent of Ki-67 positive cells to the right of the horizontal gate, Ki-67 HSSC refers to the upper right quadrant, and Ki-67 LSSC refers to the lower right quadrant.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every clement specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “biological sample,” “tissue sample,” “specimen” or the like refers to any sample including a biomolecule (such as a protein, a peptide, a nucleic acid, a lipid, a carbohydrate, or a combination thereof) that is obtained from any organism including viruses. Other examples of organisms include mammals (such as humans; veterinary animals like cats, dogs, horses, cattle, and swine; and laboratory animals like mice, rats, and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments, or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

As used herein, the terms “biomarker” or “marker” refer to any molecule or group of molecules found in a biological sample that can be used to characterize the biological sample or a subject from which the biological sample is obtained. For example, a biomarker may be a molecule or group of molecules whose presence, absence, or relative abundance is characteristic of a particular cell or tissue type or state; or characteristic of a particular pathological condition or state; or indicative of the severity of a pathological condition, the likelihood of progression or regression of the pathological condition, and/or the likelihood that the pathological condition will respond to a particular treatment. As another example, the biomarker may be a cell type or a microorganism (such as a bacterium, mycobacterium, fungus, virus, and the like), or a substituent molecule or group of molecules thereof.

As used herein, a “gate” generally refers to a set of boundary points identifying a subset of data of interest. In cytometry, a gate may bound a group of events of particular interest.

As used herein the term “gating” refers to the selection of a population of particles from a sample, based on the characteristics of the particle. For example, characteristics of a particle can be defined based on the front scatter content (FSC), side scatter content (SSC), and/or fluorescence intensity. Particles with the required characteristics will pass through the gate and are selected for further analysis, while those that do not have the required characteristics will not be selected for further analysis. Digital gating means that one or more populations (e.g., cell populations) are selected to show on a plot (e.g., a scatter plot or a histogram plot) after analyzing a mixture of all cells. The selected cell population is “digitally sorted” after analysis of all cells. Physical sorting is the process of removing the selected population from all cells into a separate tube, then analyzing the selected cell population. Both digital gating/sorting and physical sorting are believed to achieve analysis of certain cell populations as described herein.

As used herein, the terms “homogenizing” or “homogenization” refer to a process (such as a mechanical process and/or a biochemical process) whereby a biological sample is brought to a state such that all fractions of the sample are equal in composition. Representative samples (as defined above) may be prepared by removal of a portion of a sample that has been homogenized. A homogenized sample (a “homogenate”) is mixed well such that removing a portion of the sample (an aliquot) does not substantially alter the overall make-up of the sample remaining and the components of the aliquot removed is substantially identical to the components of the sample remaining. In the present disclosure the “homogenization” will in general preserve the integrity of the majority of the cells within the sample, e.g., at least 50% of the cells in the sample will not be ruptured or lysed as a result of the homogenization process. In other embodiments, homogenization will preserve the integrity of at least 80% of the cells in the sample. In other embodiments, homogenization will preserve the integrity of at least 85% of the cells in the sample. In other embodiments, homogenization will preserve the integrity of at least 90% of the cells in the sample. In other embodiments, homogenization will preserve the integrity of at least 95% of the cells in the sample. In other embodiments, homogenization will preserve the integrity of at least 96 of the cells in the sample. In other embodiments, homogenization will preserve the integrity of at least 97% of the cells in the sample. In other embodiments, homogenization will preserve the integrity of at least 98% of the cells in the sample. In other embodiments, homogenization will preserve the integrity of at least 99% of the cells in the sample. In other embodiments, homogenization will preserve the integrity of at least 99.9% of cells in the same. The homogenates may be substantially dissociated into individual cells (or clusters of cells) and the resultant homogenate or homogenates are substantially homogeneous (consisting of or composed of similar elements or uniform throughout).

As used herein, “immunohistochemistry” or “IHC” refers to a method of determining the presence or distribution of an antigen in a sample by detecting interaction of the antigen with a specific binding agent, such as an antibody. A sample including an antigen is incubated with an antibody under conditions permitting antibody-antigen binding. Antibody-antigen binding can be detected by means of a detectable moiety conjugated to the antibody (direct detection) or by means of a detectable moiety conjugated to a secondary antibody, which is raised against the primary antibody (e.g., indirect detection). In some examples, IHC is utilized to detect the presence of or determine the amount of one or more proteins in a sample. IHC is further describes in International Publication No. WO2013019945, the disclosure of which is hereby incorporated by reference in its entirety.

As used herein, “in situ hybridization” or “ISH” references to a process of contacting a sample containing a target nucleic acid or a genomic target nucleic acid with a labeled probe specifically hybridizable or specific for the target nucleic acid. In some embodiments, the labeled probe (formulated in a suitable hybridization) and the sample are combined, under conditions and for sufficient time to permit hybridization to occur (typically to reach equilibrium). The chromosome preparation is washed to remove excess probe, and detection of specific labeling of the target is performed using standard techniques. IHC is further describes in International Publication No. WO2015124702, the disclosure of which is hereby incorporated by reference in its entirety.

As used herein, the terms the terms “detectable moiety,” “reporter moiety,” “label,” or “stain” refer to a reagent that is capable of binding to an analyte, being internalized or otherwise absorbed, and being detected, e.g., through shape, morphology, color, fluorescence, luminescence, phosphorescence, absorbance, magnetic properties, or radioactive emission. Likewise, the terms “labeling,” “staining,” or the like as used herein generally refers to any treatment of a biological specimen that detects and/or differentiates the presence, location, and/or amount (such as concentration) of a particular molecule (such as a lipid, protein or nucleic acid) or particular structure (such as a normal or malignant cell, cytosol, nucleus, Golgi apparatus, or cytoskeleton) in the biological specimen. For example, staining can provide contrast between a particular molecule or a particular cellular structure and surrounding portions of a biological specimen, and the intensity of the staining can provide a measure of the amount of a particular molecule in the specimen. Staining can be used to aid in the viewing of molecules, cellular structures, and organisms not only with bright-field microscopes, but also with other viewing tools, such as phase contrast microscopes, electron microscopes, and fluorescence microscopes. Some staining performed by the system can be used to visualize an outline of a cell. Other staining performed by the system may rely on certain cell components (such as molecules or structures) being stained without or with relatively little staining other cell components. Examples of types of staining methods performed by the system include, without limitation, histochemical methods, immunohistochemical methods, and other methods based on reactions between molecules (including non-covalent binding interactions), such as hybridization reactions between nucleic acid molecules. Staining methods include, but are not limited to, primary staining methods (e.g., H&E staining, Pap staining, etc.), enzyme-linked immunohistochemical methods, and in situ RNA and DNA hybridization methods, such as fluorescence in situ hybridization (FISH).

As used herein, the term “next generation sequencing” refers to sequencing technologies having high-throughput sequencing as compared to traditional Sanger- and capillary electrophoresis-based approaches, wherein the sequencing process is performed in parallel, for example producing thousands or millions of relatively small sequence reads at a time. Some examples of next generation sequencing techniques include, but are not limited to, sequencing by synthesis, sequencing by ligation, and sequencing by hybridization. These technologies produce shorter reads (anywhere from about 25-about 500 bp) but many hundreds of thousands or millions of reads in a relatively short time. Examples of such sequencing devices available from Illumina (San Diego, CA) include, but are not limited to iSEQ, MiniSEQ, MiSEQ, NextSEQ, NoveSEQ.

It is believed that the Illumina next-generation sequencing technology uses clonal amplification and sequencing by synthesis (SBS) chemistry to enable rapid sequencing. The process simultaneously identifies DNA bases while incorporating them into a nucleic acid chain. Each base emits a unique fluorescent signal as it is added to the growing strand, which is used to determine the order of the DNA sequence. A non-limiting example of a sequencing device available from ThermoFisher Scientific (Waltham, MA) includes the Ion Personal Genome Machine™ (PGM™) System.

It is believed that Ion Torrent sequencing measures the direct release of H+ (protons) from the incorporation of individual bases by DNA polymerase. A non-limiting example of a sequencing device available from Pacific Biosciences (Menlo Park, CA) includes the PacBio Sequel Systems. A non-limiting example of a sequencing device available from Roche (Pleasanton, CA) is the Roche 454. Next-generation sequencing methods may also include nanopore sequencing methods. In general, three nanopore sequencing approaches have been pursued: strand sequencing in which the bases of DNA are identified as they pass sequentially through a nanopore, exonuclease-based nanopore sequencing in which nucleotides are enzymatically cleaved one-by-one from a DNA molecule and monitored as they are captured by and pass through the nanopore, and a nanopore sequencing by synthesis (SBS) approach in which identifiable polymer tags are attached to nucleotides and registered in nanopores during enzyme-catalyzed DNA synthesis. Common to all these methods is the need for precise control of the reaction rates so that each base is determined in order.

Strand sequencing requires a method for slowing down the passage of the DNA through the nanopore and decoding a plurality of bases within the channel; ratcheting approaches, taking advantage of molecular motors, have been developed for this purpose. Exonuclease-based sequencing requires the release of each nucleotide close enough to the pore to guarantee its capture and its transit through the pore at a rate slow enough to obtain a valid ionic current signal. In addition, both methods rely on distinctions among the four natural bases, two relatively similar purines and two similar pyrimidines.

The nanopore SBS approach utilizes synthetic polymer tags attached to the nucleotides that are designed specifically to produce unique and readily distinguishable ionic current blockade signatures for sequence determination. In some embodiments, sequencing of nucleic acid molecules comprises via nanopore sequencing comprises preparing nanopore sequencing complexes and determining polynucleotide sequences. Methods of preparing nanopores and nanopore sequencing are described in U.S. Patent Application Publication No. 2017/0268052, and PCT Publication Nos. WO2014/074727, WO2006/028508, WO2012/083249, and WO/2014/074727, the disclosures of which are hereby incorporated by reference herein in their entireties. In some embodiments, tagged nucleotides may be used in the determination of the polynucleotide sequences (see, e.g., PCT Publication No. WO/2020/131759, WO/2013/191793, and WO/2015/148402, the disclosures of which are hereby incorporated by reference herein in their entireties).

Analysis of the data generated by sequencing is performed using software and/or statistical algorithms that perform various data conversions, e.g., conversion of signal emissions into base calls, conversion of base calls into consensus sequences for a nucleic acid template, etc. Such software, statistical algorithms, and the use of such are described in detail, in U.S. Patent Application Publication Nos. 2009/0024331 2017/0044606 and in PCT Publication No. WO/2018/034745, the disclosures of which are hereby incorporated by reference herein in their entireties.

As used herein, the terms “primary antibody” and “secondary antibody” refer to different antibodies, where a primary antibody is a polyclonal or monoclonal antibody from one species (rabbit, mouse, goat, donkey, etc.) that specifically recognizes an antigen (e.g., a biomarker) in a sample (e.g., a human biological sample) under study, and a secondary antibody is an antibody (usually polyclonal) from a different species that specifically recognizes the primary antibody, e.g., in its Fc region.

As used herein, the terms “representative sample” and “representative sampling” as used herein refer to a sample (or a subset of a sample) that accurately reflects the components of the entirety and, thus, the sample is an unbiased indication of the entire population. In general, this means that the different types of cells and their relative proportion or percentages within the representative sample or a portion thereof essentially accurately reflects or mimics the relative proportion or percentages of these cell types within the entire tissue specimen, generally a solid tumor or portion thereof. Sampling is the operation of securing portions of an object for subsequent analysis. Representative samples are generated in a way that a reasonably close knowledge of the object being studied can be obtained. By contrast, conventional random sampling methods, generally does not give rise to a “representative sample.” While the selection of smaller individual sub-samples from a larger sample can be biased based on the regions selected, homogenizing a large sample, e.g., an entire tumor or lymph node, results in spatially segregated elements being homogenously dispersed throughout the sample.

As used herein, the term “sequencing” refers to the determination of the order and position of bases in a nucleic acid molecule. More particularly, the term “sequencing” refers to biochemical methods for determining the order of the nucleotide bases, adenine, guanine, cytosine, and thymine, in a DNA oligonucleotide. Sequencing, as the term is used herein, can include without limitation parallel sequencing or any other sequencing method known of those skilled in the art, for example, chain-termination methods, rapid DNA sequencing methods, wandering-spot analysis, Maxam-Gilbert sequencing, dye-terminator sequencing, or using any other modern automated DNA sequencing instruments.

As used herein, the terms “stratifying” and “classifying” are used interchangeably herein to refer to sorting of subjects into different strata or classes based on the features of a particular physiological or pathophysiological state or condition. For example, stratifying a population of subjects according to whether they are likely to respond to a therapy (e.g., chemotherapy or immunotherapy) involves assigning the subjects based on levels of response to therapy biomarkers.

As used herein, the term “tumor” refers to all neoplastic cell growth and proliferation, whether malignant or benign, and all pre-cancerous and cancerous cells and tissues. In some embodiments, the tumor is a malignant cancerous tumor (i.e., cancer). In some embodiments, the tumor is a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, prolymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, colorectal (e.g., basaloid colorectal carcinoma), breast, prostate, lung, kidney, liver, pancreas, ovary (e.g., endometrioid ovarian carcinoma), head and neck, oral cavity, stomach, duodenum, small intestine, large intestine, anus, gall bladder, labium, nasopharynx, skin, uterus, male genital organ, urinary organs (e.g., urothelium carcinoma, dysplastic urothelium carcinoma, transitional cell carcinoma), bladder, and skin. Solid tumors of non-epithelial origin include sarcomas, brain tumors, and bone tumors.

As used herein, the term “tumor sample” encompasses samples prepared from a tumor or from a sample potentially comprising or suspected of comprising cancer cells, or to be tested for the potential presence of cancer cells, such as a lymph node. As used herein, the term “tumor” refers to a mass or a neoplasm, which itself is defined as an abnormal new growth of cells that usually grow more rapidly than normal cells and will continue to grow if not treated sometimes resulting in damage to adjacent structures. Tumor sizes can vary widely. A tumor may be solid, or fluid filled. A tumor can refer to benign (not malignant, generally harmless), or malignant (capable of metastasis) growths. Some tumors can contain neoplastic cells that are benign (such as carcinoma in situ) and, simultaneously, contain malignant cancer cells (such as adenocarcinoma). This should be understood to include neoplasms located in multiple locations throughout the body. Therefore, for purposes of the disclosure, tumors include primary tumors, lymph nodes, lymphatic tissue, and metastatic tumors.

### Overview

The present disclosure is directed to cytometrically assaying a sample, e.g., a labeled cell suspension and, in particular, cytometrically assaying labeled cells in one or more aliquots derived from a sample (e.g., a homogenized fixed tissue sample, a sample comprising dissociated fixed cells, or a representative sample). In some embodiments, the present disclosure is directed to a cytometric assay for distinguishing between tumor cells expressing a cell proliferation marker (e.g., Ki-67) and normal cells expressing the cell proliferation marker (e.g., Ki-67). In some embodiments, the present disclosure is also directed to quantifying a percentage of normal cells expressing a cell proliferation marker (e.g., Ki-67) and a percentage of tumor cells expressing the cell proliferation marker (e.g., Ki-67). By having an understanding of the percentage of tumor cells staining positive for a cell proliferation marker versus the percentage of normal cells staining positive for the cell proliferation maker, patients in need of treatment with adjuvant therapy or neoadjuvant therapy may be stratified into one or more populations, such as a first population that would likely receive a benefit from the adjuvant therapy or the neoadjuvant therapy; and a second population that would less likely receive a benefit from the adjuvant therapy or the neoadjuvant therapy.

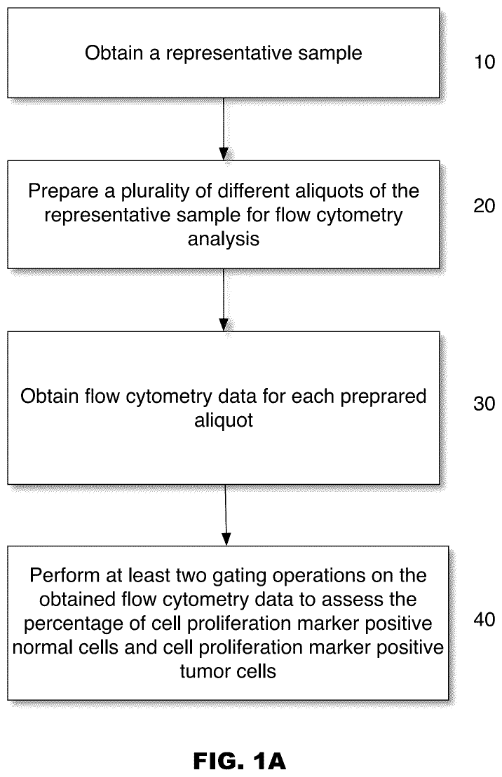

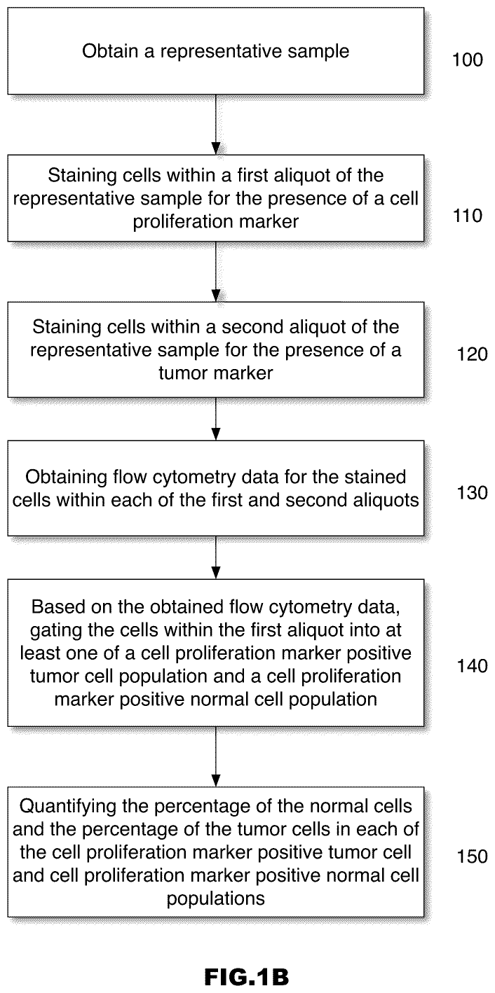

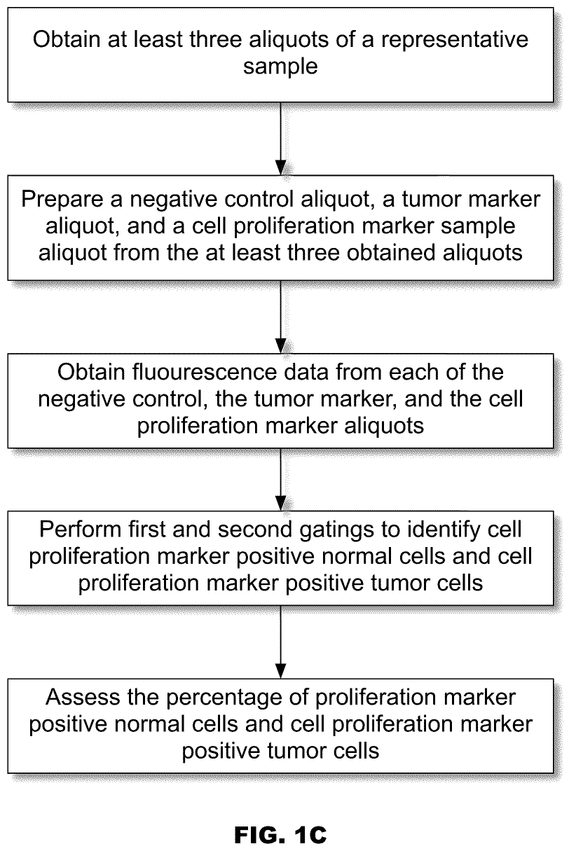

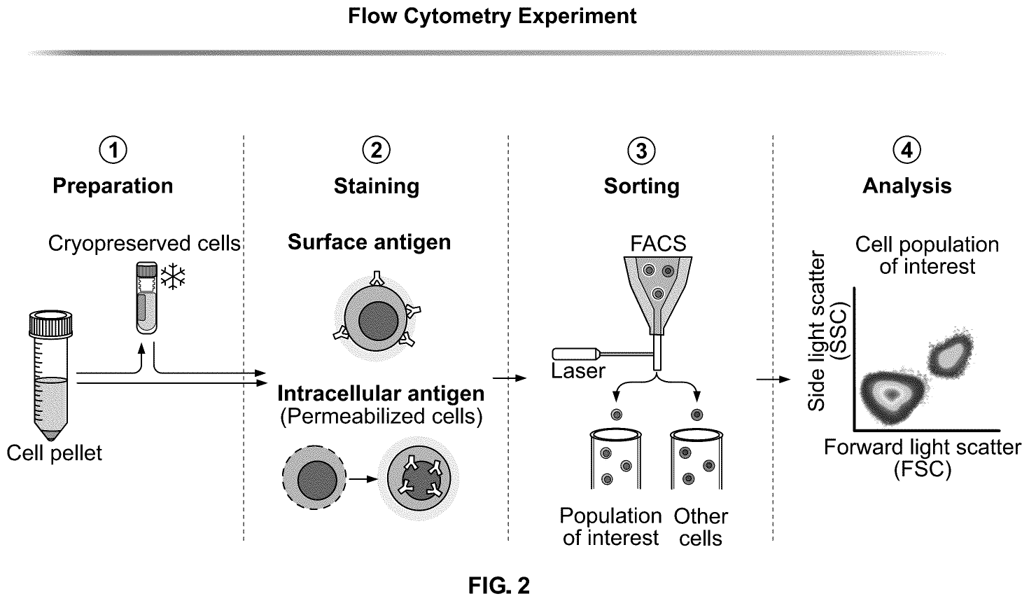

FIGS. 1A, 1B, 1C, and 2 illustrate methods of conducting a cytometric assay on a sample in accordance with some embodiments of the present disclosure. First, a sample is obtained (steps 10 and 100). In some embodiments, the sample is derived from one or more tumor samples, such as one or more tumor samples derived from a patient in need of adjuvant therapy or neoadjuvant therapy. In some embodiments, the sample is derived from one or more surgical tissue resections. In other embodiments, the sample is derived from residual surgical material, such as surgical material remaining after surgical biopsies are obtained.

Subsequently, one or more aliquots derived from the obtained sample are prepared for cytometric analysis (FIG. 1A, step 20). In some embodiments, the obtained sample is divided into a plurality of aliquots and each aliquot is stained for the presence of one or more biomarkers (e.g., Ki-67, cytokeratin, CD3, etc.) and optionally counterstained for the presence of DNA (e.g., counterstained with DAPI). By way of example, in some embodiments, cells within a first aliquot derived from the obtained sample are stained for the presence of a cell proliferation marker (step 110) and optionally counterstained. Likewise, in some embodiments, cells within a second aliquot derived from the obtained sample are stained for the presence of at least a tumor marker (step 120) and optionally counterstained. In some embodiments, one or more additional aliquots derived from the sample are stained for the presence of other biomarkers, e.g., lineage specific markers, markers of normal cells, markers of immune cells; and/or optionally counterstained for the presence of DNA. In some embodiments, yet additional aliquots derived from the representative sample are prepared as negative controls. As described herein, the cells within any negative control may be incubated with one or more detection reagents (e.g., secondary antibodies, fluorophores, etc.) and optionally counterstained (e.g., with DAPI).

Next, cytometry data is obtained for each prepared aliquot, i.e., cytometry data is obtained for the stained cells within each of the aliquots derived from the sample and/or negative control aliquot (steps 30 and 130). In some embodiments, the obtained cytometry data comprises scatter plots of fluorescence intensity versus side scatter content (SSC). In other embodiments, the obtained cytometry data comprises histogram plots of DNA content.

Subsequently, one or more gating operations are performed (steps 40 and 140) to classify normal cells expressing a cell proliferation marker into a first population and tumor cells expressing a cell proliferation marker into a second population. In some embodiments, the method further comprises quantifying the percentage of the normal cells expressing the cell proliferation marker and the percentage of the tumor cells expressing the cell proliferation marker in each of the cell proliferation marker positive tumor cell and cell proliferation marker positive normal cell populations (step 150).

In some embodiments, and with reference to FIG. 2, cells may be obtained from a sample (step 1). Subsequently, those cells may be stained for the presence of one or more markers (step 2). In some embodiments, a plurality of aliquots is obtained from a single sample, and each aliquot of the plurality of aliquots is stained for the presence of one or more biomarkers (e.g., a cell proliferation maker, a tumor cell marker, an immune cell marker, a marker found in normal cells). Next, the stained cells are analyzed using a flow cytometer and fluorescence data is acquired (see, e.g., the scatter plot of SSC versus FSC depicted at step 4). Optionally, the stained cells are sorted using the flow cytometer (step 3).

In some embodiments, the one or more gated populations of stained cells may be subsequently further analyzed or treated in one or more downstream operations. In some embodiments, the one or more gated populations of cells may be physically sorted (e.g., such as by size; such as using one or more microfluidic devices). In other embodiments, the one or more gated populations of cells may be further stained (in a simplex or multiplex assay) for the presence of one or more additional biomarkers (and/or subject to further cytometric analysis). In some embodiments the one or more gated populations or cells may be subjected to image analysis. In some embodiments, an image analysis algorithm and/or system may be utilized that automatically compute a score from a set of images of multiplex IHC slides and/or fluorescent stained slides derived from one or more aliquots of the representative sample.

In some embodiments, the method further comprises sequencing isolated genomic material from one or more cell populations of interest. For instance, and in some embodiments, isolated genomic material (e.g., DNA, RNA) from either the cell proliferation marker positive normal cell population and/or the cell proliferation marker positive tumor cell population may be sequenced, such as with a next-generation sequencing platform or with a single cell sequencing platform. By way of example, and with reference to FIG. 2 (step 3), flow cytometry data may be utilized to sort stained cells into a specific population of interest (e.g., cell proliferation marker positive tumor cells), genomic material may be isolated from the sorted specific population of interest, and then the isolated genomic material may be sequenced.

Single-cell sequencing technologies refer to the sequencing of a single-cell genome or transcriptome, so as to obtain genomic, transcriptome or other multi-omics information to reveal cell population differences and cellular evolutionary relationships. As compared with next-generation sequencing, single cell sequencing measures the genomes of individual cells in a cell population. Compared with traditional sequencing technology, single-cell technologies have the advantages of detecting heterogeneity among individual cells, distinguishing a small number of cells, and delineating cell maps. Single cell sequencing technologies can measure different types of genetic material—the genome, the transcriptome or the methylome—of a single cell.

In some embodiments, genomic material is extracted from single cells, such as single cells from the one or more populations of single cells prepared after cell sorting. In some embodiments, genomic material is amplified within isolated individual cells. In some embodiments, the extracted genomic material is amplified, optionally barcoded, and a sequencing library is prepared including the genomic material from an isolated single cell. The sequencing library is then sequenced, such as with a next-generation sequencing apparatus.

In some embodiments, the single cell sequencing includes single cell genome sequencing (scDNA-seq), which facilitates the genomic heterogeneity of a cellular population to be studied. In some embodiments, the single cell sequencing includes single cell transcriptome sequencing (scRNA seq). In some embodiments, the single cell sequencing includes single cell DNA methylome sequencing (scDNA-Met-seq). Methylation is an epigenetic mechanism that changes the DNA activity without affecting its sequence. In some embodiments, scDNA-Met-seq is utilized to study the epigenetic changes within an otherwise genetically identical cellular population.

### Samples

In some embodiments, the samples of the present disclosure are derived from one or more tumor samples or biopsy samples; and/or derived from residual surgical material. In some embodiments, the sample is derived from a fixed tissue specimen, e.g., a tissue specimen fixed in 10% neutral buffered formalin or another aldehyde-based fixative. As such, in some embodiments, the samples of the present disclosure are derived from one or more fixed tumor samples or fixed biopsy samples; and/or derived from fixed residual surgical material. In other embodiments, the sample is derived from a fixed tissue specimen, but not one that has been embedded in paraffin, e.g., the sample may be fixed but not embedded in paraffin or derived from a paraffin-embedded sample. In further embodiments, the sample is derived from a formalin-fixed paraffin-embedded tissue specimen or cytology specimen.

In some embodiments, the sample comprises a homogenized fixed tissue sample, e.g., one that has been mechanically blended. In other embodiments, the sample comprises dissociated cells derived from a fixed tissue sample or from a homogenized fixed tissue sample. In yet other embodiments, the sample is a representative sample, as described herein. In some embodiments, the homogenize sample may be further dissociated as described herein.

### Preparation of a Representative Sample

The present disclosure provides methods of forming a representative sample for use in flow cytometry analysis.

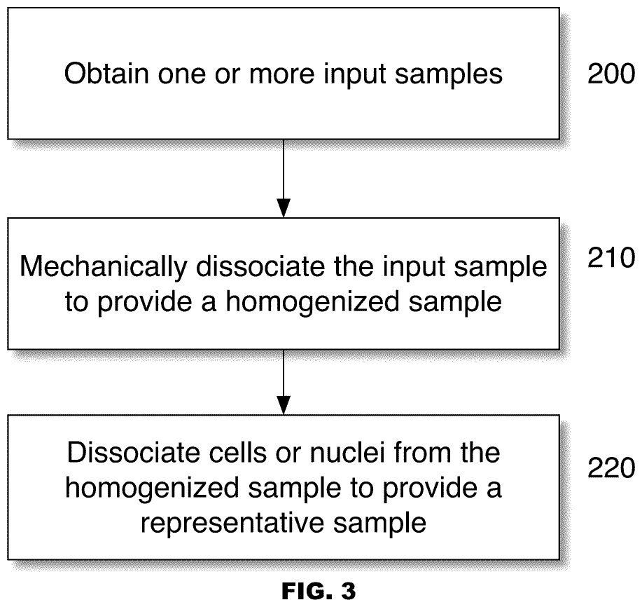

With reference to FIG. 3, in some embodiments one or more input samples, e.g., one or more fixed or unfixed tumor samples, one or more fixed or unfixed biopsy samples, or fixed or unfixed residual surgical material, are obtained (step 200); and then the one or more input samples are homogenized to form a homogenate (step 210). Cells within the homogenate are then dissociated to form the representative sample (step 220). Following sufficient dissociation of the input sample, the subpopulations of cells (including tumor cells) that were originally spatially segregated within the original sample are substantially uniformly distributed throughout the representative sample. That is, as a result of homogenizing the input sample, any heterogeneity of cells within the input sample is substantially uniformly distributed within the resulting representative sample or a portion or fraction thereof, such that the representative sample (or any portion or fraction thereof) substantially uniformly expresses the heterogeneity of the input sample (or one or more input samples) from which it was derived.

In some embodiments, and as a result of homogenizing the input sample, any aliquot removed from the representative sample comprises one or more populations of subclones at a proportion (or ratio) at which they existed within the input sample. Different tumor cell populations that arise as a result of tumor heterogeneity are called “subclones,” the progeny of a mutant cell arising in a clone. The prevalence of subclones within a tumor may vary. Certain subclones comprise the majority of the tumor but decrease over time and/or following certain treatments. Other subclones are initially undetectable, but later become abundant. Multiple subclones can exists simultaneously and vary in their prevalence over time it takes for the tumor to grow large enough to be detectable. The term “low prevalence events” or “low prevalence genetic events” within a tumor refers to rare events or rare genetic events (such as mutations) that occur at a rate of about 10 to about 1%, about 1 to about 0.1%, about 0.1 to about 0.01%, about 0.01 to about 0.001%, about 0.001 to about 0.0001%, about 0.0001 to about 0.00001%, about 0.00001 to about 0.000001%, or below about 0.000001%. Because the sample generated by the disclosed methods is representative (or substantially representative) of the tumor as a whole, even low prevalence subclones (such as down to at least about 0.000001%) in a tumor or biological sample can be detected, in addition to all other subclones that exist at higher prevalence rates. In some embodiments, any aliquot removed from the representative sample comprises one or more populations of low prevalence subclones at the proportion at which they existed within the input sample.

### Input Sample

In some embodiments, the representative sample is derived from a tumor (cancerous or non-cancerous), a metastatic lesion, normal tissue, whole blood, a lymph node, or any combination therefore (collectively the “input sample”). In some embodiments, the representative sample is derived from a surgical resection or residual surgical material (such as material from which biopsies have been collected which would otherwise be destroyed). In some embodiments, the representative samples disclosed herein are obtained by homogenization and dissociation of one or more putative normal tissue specimens, e.g., derived from a subject at risk of developing cancer as the result of a genetic mutation or prior cancer, or adjacent normal tissue from a surgical resection for use as a control sample. Any of these samples may be fixed (such as using one or more aldehyde-based fixatives).

In some embodiments, the residual surgical tumor sample is a specimen that would have otherwise been destroyed. In some embodiments, an entire obtained residual surgical tumor sample is fixed in an aldehyde-based fixative but not paraffin embedded, and wherein all the fixed but not paraffin embedded residual surgical sample is homogenized. In some embodiments, an entire obtained residual surgical tumor sample is fixed in an aldehyde-based fixative, and wherein all the fixed residual surgical sample is homogenized. In some embodiments, residual surgical tumor sample is not derived from tissue specimens used for diagnostic purposes. In some embodiments, residual surgical tumor sample is not derived from tissue specimens used TNM staging.

In some embodiments, the input sample (e.g., the tumor or other tissue sample utilized) has have a volume of at least about 1 cm³, at least about 2 cm³, at least about 3 cm³, at least about 4 cm³, at least about 5 cm³, at least about 6 cm³, at least about 7 cm³, at least about 8 cm³, at least about 9 cm³, at least about 10 cm³, at least about 15 cm³, at least about 20 cm³, at least about 25 cm³, at least about 50 cm³, at least about 100 cm³, at least about 250 cm³, at least about 500 cm³, at least about 1,000 cm³, at least about 2,500 cm³, at least about 5,000 cm³, at least about 7,500 cm³, at least about 10,000 cm³ or larger. In some embodiments, the input sample (e.g., the tumor or other tissue sample utilized) may have a width at the widest point of at least about 0.5 cm, at least about 1 cm, at least about 1.5 cm, at least about 2 cm, at least about 2.5 cm, at least about 3 cm, at least about 3.5 cm, at least about 4 cm, at least about 4.5 cm, at least about 5 cm, at least about 6 cm, at least about 7 cm, at least about 10 cm, at least about 25 cm, at least about 50 cm or larger.

In some embodiments, the input sample comprises at least about 100-about 200; 200-about 1,000; about 1,000-about 5,000; about 10,000-about 100,000; about 100,000-about 1,000,000; about 1,000,000-about 5,000,000; about 5,000,000-about 1,000,000,000; about 1,000,000,000-about 5,000,000,0000, or more cells, optionally from spatially distinct regions of the tumor. Generally, there are about 1 billion cells in a tumor or portion thereof having an about 1 cm diameter and, for the most part, this relationship proceeds on a linear scale. For example, an excisional sample such as a biopsy having about a 2 cm diameter can comprise about 3-about 5 billion cells.

In some embodiments, the representative sample is derived from a fresh tissue sample, namely one that has not been preserved. In other embodiments, the representative sample is derived from a fixed tissue sample, e.g., a tissue sample fixed in about 10% neutral buffered formalin. In yet other embodiments, the representative sample is derived from a fixed tissue sample, but not one that has been embedded in paraffin, e.g., the representative sample may be fixed but not embedded in paraffin or derived from a paraffin-embedded sample. In further embodiments, the representative sample is derived from a formalin-fixed paraffin-embedded sample.

### Homogenization

Methods of preparing a homogenate from one or more input tissue samples are described in United States Patent Publication No. 2020/0049599, the disclosure of which is hereby incorporated by reference herein in its entirety. For instance, and in some embodiments, a tumor sample (e.g., a surgical resection or residual surgical material), lymph node sample, blood sample, and/or other tissue sample is homogenized through a mechanical dissociation method, such as by placing the input sample into a mechanical shearing apparatus, e.g., a blender or an ultra sonicator. Alternatively, the input sample may be chemically dissociated, such as through enzymatic dissociation. In yet other embodiments, the input sample may be homogenized through any combination of mechanical dissociation and chemical dissociation. For instance, and with reference to FIG. 3B, in some embodiments, the formation of a representative sample comprises mechanically dissociating (or shearing) an obtained input sample to provide a homogenized sample (step 210); and then dissociating cells or nuclei from the homogenized sample to provide the representative sample (step 220).

“Mechanical dissociation” refers to a homogenization of an obtained input tissue sample using one or more mechanical sources and/or with one or more sources that each apply physical forces to the obtained tissue sample. In some embodiments, the one or more mechanical homogenization techniques include applying physical forces onto the obtained tissue sample such as shearing, slicing, cutting, vibration, pressure, crushing to pull apart or tear or otherwise separate the tissue sample into smaller pieces. In other embodiments, the one or more mechanical homogenization techniques include homogenizing, shearing, cutting, mincing, scraping, or scratching the obtained tissue sample. In some embodiments, the at least one mechanical homogenization process comprises the use of a mortar and pestle, a dounce homogenizer, a tissue grinder, a Waring blender, a mortar, and pestle, triuration with a glass Pasteur pipette, vortexing, a hand-held electronic rotary blade tissue homogenizer, or a bead beating homogenizer. In some embodiments, each mechanical dissociation process may be performed for a period of time ranging from between about 5 seconds to about 5 minutes, such as from about 5 seconds to about 4 minutes, from about 10 seconds to about 3 minutes, from about 10 seconds to about 2 minutes, etc.

In some embodiments, mechanical homogenization may take place in several steps, where during each step the size, weight, and/or complexity of the mechanically homogenized sample is further reduced. For instance, in some embodiments, and depending on the type of mechanical homogenization technique applied, an initial mechanical homogenization process may result in a mechanically homogenized sample which includes a mixture of cell clusters, clumps of cells, and/or individual cells. In these embodiments, the mechanically homogenized sample comprising the clusters, clumps, and/or individual cells may then be further mechanically homogenized using one or more mechanical homogenization processes to provide a further homogenized solution, i.e., one where the size, weight, and/or complexity of any clusters and/or clumps of cells is reduced.

In some embodiments, mechanical dissociation may take place in several steps, where during each step the size, weight, and/or complexity of the mechanically dissociated sample is further reduced. For instance, in some embodiments, and depending on the type of mechanical dissociation technique applied, an initial mechanical dissociation process may result in a mechanically dissociated sample which includes a mixture of cell clusters, clumps of cells, and/or individual cells. In these embodiments, the mechanically dissociated sample comprising the clusters, clumps, and/or individual cells may then be further mechanically dissociated using one or more mechanical dissociation processes to provide a further dissociated solution, i.e., one where the size, weight, and/or complexity of any clusters and/or clumps of cells is reduced.

In some embodiments, homogenization may be effectuated by a method which preserves the integrity of the cells within the sample, i.e., the bulk of the cells within the homogenized sample or samples are not lysed and whereby the resultant homogenate and portions thereof are “representative” of the sample or samples. Therefore, the cells within the sample or a portion thereof reflect the percentages of the different cell types within the entirety of the tissue sample or samples, e.g., a solid tumor or a lymph node. This may be accomplished, for example, by mechanical dissociation of the tumor sample or a portion thereof (such as mechanical dissociation performed with or without the addition of liquid to the tumor sample or a portion thereof) and/or chemical or enzymatic dissociation of the tumor sample or a portion thereof (such as treatment with an enzyme that selectively or preferentially or primarily acts upon extracellular matrix proteins as compared to membrane-associated proteins). Alternatively, homogenization of the input sample may result in the dissociation of the cells from the input sample while still generating a sample that is representative of the starting tissue, such as a whole tumor. In some embodiments, the homogenized input sample may optionally be further dissociated and/or treated to remove or isolate specific types of molecules such as specific cell types, proteins, nucleic acids, or lipids, and the like thereby generating other representative samples which may be used in diagnostic and therapeutic methods.

In some embodiments, mechanical dissociation comprises cutting, scraping, or scratching the tissue into small pieces. In some embodiments, the minced tissue is washed in medium to separate the cells from the tissue. Optionally, the minced tissued may be agitated and/or sonicated are to loosen the cells. In some embodiments, the homogenization processes comprise the use of a mechanical process, non-limiting examples of such include mortar and pestle, a dounce homogenizer or tissue grinder, a hand held electronic rotary blade tissue homogenizer (such as Omni-TH available from Thomas Scientific), a bead beating homogenizer (such as a bullet blender or a Burton Precellys 24 Tissue Homogenizer or a Bead Ruptor available from OMNI), optionally at a speed of between about 100 and about 75,000 RPM for rotational homogenizers or a speed of about 0.5 m/s to about 2.5 m/s for bead beaters, and for a length of about 30 second to about 5 minutes, about 5 minutes to about 10 minutes, about 10 minutes to about 30 minutes, or about 30 minutes to about 60 minutes. In some embodiments, the homogenate is created by manual dicing using a scalpel or knife. In some embodiments, the homogenization further comprises cell conditioning, where the cell conditioning comprises adjusting pH and/or heat, and subsequently treating the sample with a cell conditioning buffer.

Enzymatic dissociation is the process of using enzymes to digest tissue pieces thereby releasing cells from tissue. Many different types of enzymes may be used in this process and, as the skilled artisan will appreciate, certain enzymes are more effective with certain tissue types. The skilled artisan will also appreciate any enzymatic dissociation process may use one or more enzymes in combination with each other, or one or more enzymes in combination with other chemical and/or mechanical dissociation methods. Non-limiting examples of suitable enzymes include, but are not limited to, collagenase, trypsin, elastase, hyaluronidase, papain, DNase I, neutral protease, and trypsin inhibitor. In some embodiments, the enzyme is selected from a group consisting of interstitial collagenase, Gelatinase-A, Stromelysin 1, Matrilysin, Neutrophil collagenase, Gelatinase-B, Stromelysin 2, Stromelysin 3, Macrophage metalloelastase, Collagenase 3, MT1-MMP, MT2-MMP, MT3-MMP, MT4-MMP, Collagenase 4, Enamelysin, X-MMP, CA-MMP, MT5-MMP, MT6-MMP, Matrilysin-2, MMP-22, endoproteinase, trypsin, chymotrypsin, endoproteinase Asp-N, endoproteinase Arg-C, endoproteinase Glu-C (V8 protease), endoproteinase Lys-C, pepsin, thermolysin, elastase, papain, proteinase K, subtilisin, clostripain, exopeptidase, carboxypeptidase A, carboxypeptidase B, carboxypeptidase P, carboxypeptidase Y, cathepsin C, acylamino-acid-releasing enzyme, and pyroglutamate aminopeptidase.

Collagenase is a proteolytic enzyme used to digest proteins found in the extracellular matrix. Unique to enzymatic proteases, collagenase can attack and degrade the triple-helical native collagen fibrils that are commonly found in connective tissue. There exist four basic collagenase types, namely: Type 1, which is suitable for use in epithelial, liver, lung, fat and adrenal tissue cell specimens; Type 2, which is suitable for use in heart, bone, muscle, thyroid and cartilage tumor originating tissues given its high proteolytic activity; Type 3, which is suitable for use in mammary cells given its low proteolytic activity; and Type 4: which is suitable for islets and other research protocols where receptor integrity is important, given its tryptic activity.

Trypsin is described as a pancreatic serine (an amino acid) protease that has specificity for peptide bonds that involve the carboxyl group of arginine and lysine amino acids. It is considered one of the most highly specific proteases. Trypsin alone is not usually effective for tissue dissociation because it shows minimal selectivity to extracellular proteins. It is usually combined with other enzymes such as collagenase or elastase.

Elastase is another pancreatic serine protease, which has specificity for peptide bonds that are next to neutral amino acids. It is unique among proteases in its ability to hydrolyze native elastin. Elastase can also be found in blood components and bacteria. In some embodiments, it is suitable for isolation of Type II cells from lung tissue.

Hyaluronidase is a polysaccharidase, this enzyme is often used for dissociation of tissues, typically when combined with a cruder protease such as collagenase. It has affinity for bonds found in about all connective tissues.

Papain is a sulfhydryl protease, it has wide specificity and so can degrade most protein substrates more thoroughly than pancreatic proteases, i.e., trypsin or elastase. Papain is frequently used to isolate neuronal materials from tissues.

Deoxyribonuclease I (DNase I) is frequently included in enzymatic cell isolation procedures to digest nucleic acids that leak into the dissociation medium and can increased viscosity and recovery problems. Without wishing to be bound by any theory, it is believed that DNasel will not damage intact cells.

Neutral protease, such Dispase® (available from Worthington Biochemical), is a bacterial enzyme with mild proteolytic activity, Dispase® is useful for isolating primary and secondary cell cultures because of its ability to maintain cell membrane integrity. It has been found to more efficiently dissociate fibroblast-like cells as compared to epithelial-like cells. It is inhibited by EDTA.

A trypsin inhibitor is derived from the soybean, it inactivates trypsin, and so is sometimes used for specific cell isolation protocols.

### Dissociation

In some embodiments and depending on the mechanical and/or biochemical dissociation process applied to the sample to generate the representative sample, the cell clusters may comprise more than one cell to thousands of cells. In some embodiments, the clusters can be dissociated (decreased in size and/or number of cells contained therein) by the application of further processing methods, e.g., by further mechanical and/or biochemical dissociation and/or by agitation, sonication, centrifugation, lateral flow processes, size exclusion, etc. depending on the subsequent assay to be performed using the representative sample (for example, IHC requiring cell clusters containing tens to thousands of cells, or FACS or flow cytometry requiring single cells or fragments of cells).

### Optional Steps

In some embodiments, the preparation of the representative sample further comprises filtering or sizing the homogenate or a portion or fraction thereof, which may result in obtaining single cells or small cell clusters, such as doublets or triplets.

In some embodiments, the cellular componentry of the representative sample may optionally be separated by one or multiple filtration steps. For example, following homogenization and optional disassociation of the homogenate through physical and/or biochemical means, the disassociated sample may be optionally filtered through a filter to remove all intact cellular material (e.g., an about 1-micron filter to an ably 100-micron filter). In other embodiments, the one or more meshes or one or more filters may have a pore size less than 100 microns. In other embodiments, the one or more meshes or one or more filters may have a pore size less than 80 microns. In other embodiments, the one or more meshes or one or more filters may have a pore size less than 70 microns. In other embodiments, the one or more meshes or one or more filters may have a pore size less than 60 microns. In other embodiments, the one or more meshes or one or more filters may have a pore size less than 50 microns. In other embodiments, the one or more meshes or one or more filters may have a pore size less than 40 microns. In other embodiments, the one or more meshes or one or more filters may have pore size less than 30 microns. In other embodiments, the one or more meshes or one or more filters may have a pore size less than 20 microns. In other embodiments, the one or more meshes or one or more filters may have a pore size less than 10 microns. In some embodiments, a series of meshes or filters ranging in size from about 1 micron to about 150 microns is used to separate cells within the homogenate.

To the extent that single cells derived from the representative sample are obtained following filtration, such cells may be analyzed using fluorescent activated cell sorting (FACS) and flow cytometry analysis (such as described herein).

### Preparing Aliquots Derived From the Representative Sample for Cytometric Analysis

The present disclosure also provides for methods of preparing one or more aliquots derived from the sample for cytometric analysis, e.g., flow cytometry (see FIG. 1A, step 20). In some embodiments, two or more aliquots are prepared for cytometric analysis. In other embodiments, three or more aliquots are prepared for cytometric analysis. In some embodiments, the one or more aliquots derived from the sample are prepared for cytometric analysis by staining cells within the one or more of the aliquots (see, e.g., steps 110 and 120 of FIG. 1B). In some embodiments, the stain is any detectable label or reporter moiety that can identify different cell types (and/or DNA) through cytometric analysis (such as with flow cytometry), for example a fluorescent label. As described herein, the cells within any aliquot may be stained for the presence of one or more cell proliferation markers, one or more tumor markers, and/or one or more normal cell markers. In some embodiments, the cells may be optionally counterstained. In some embodiments, one or more negative control aliquots, derived from the sample, are also prepared for flow cytometry analysis.

In some embodiments, one or more aliquots of the sample are obtained, and cells within each of the one or more aliquots are stained for the presence of one or more different biomarkers and/or counterstained. In some embodiments, at least two aliquots of the sample are obtained (e.g., a tumor marker aliquot; and a proliferation marker aliquot), and cells within each of the at least two aliquots are stained for the presence of one or more different biomarkers and/or counterstained. In some embodiments, at least three aliquots of the sample are obtained (e.g., a tumor marker aliquot; a proliferation marker; and a negative control aliquot), and cells within each of the at least three aliquots are stained for the presence of one or more different biomarkers and/or counterstained. In some embodiments, at least four aliquots of the representative sample are obtained (e.g., a tumor marker aliquot; a proliferation marker aliquot; a negative control aliquot; and a normal marker aliquot), and cells within each of the at least four aliquots are stained for the presence of one or more different biomarkers and/or counterstained.

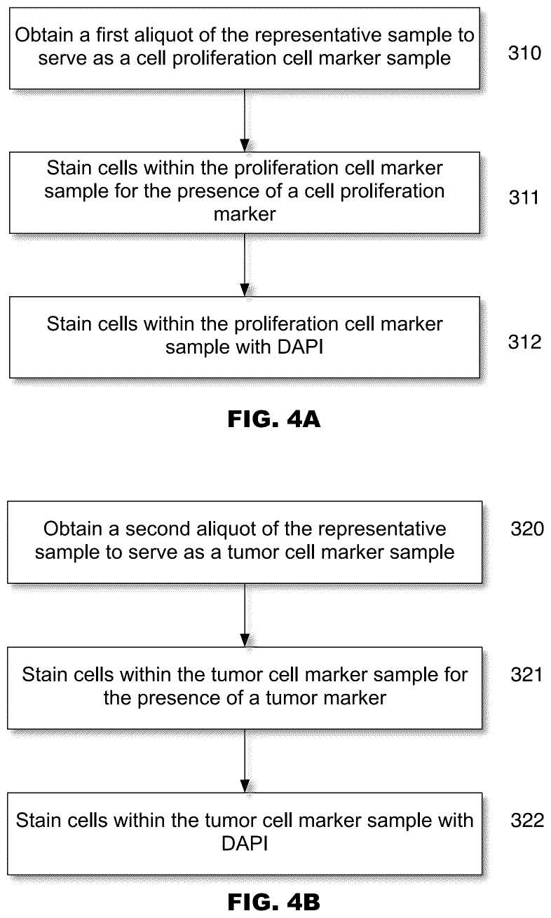

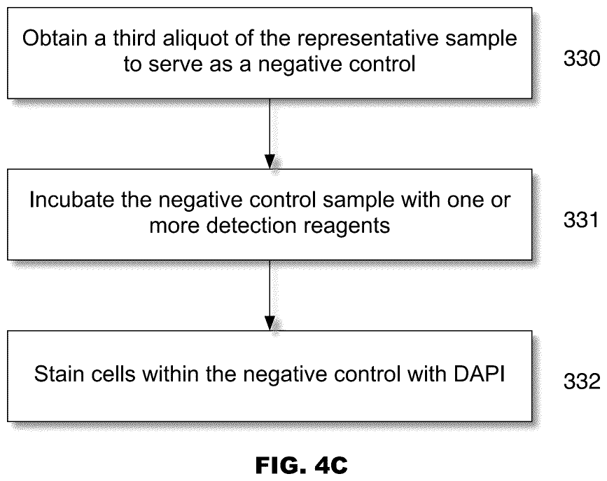

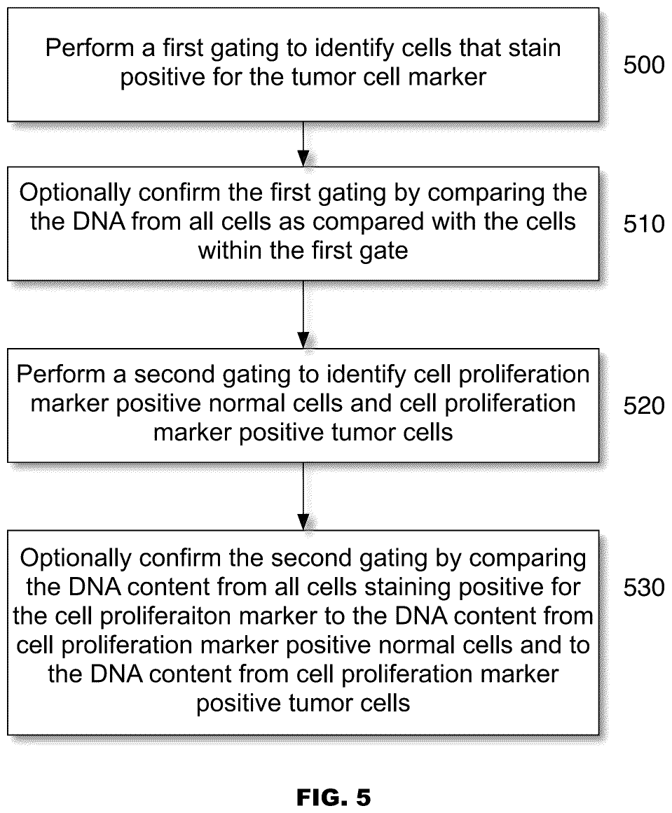

In some embodiments, at least three aliquots of the sample are obtained and cells within at least two of the three aliquots are stained for the presence of different biomarkers. For instance, and with reference to FIG. 4A, cells within a first aliquot (step 310) of the at least three aliquots are stained (e.g., fluorescently stained) (step 311) for the presence of one or more cell proliferation markers (e.g., Ki-67) and optionally counterstained (e.g., to identify DNA, such as with 4′,6-diamidino-2-phenylindole (DAPI) (step 312). This provides for a proliferation marker aliquot (see FIG. 1C). In some embodiments, cells within a second aliquot (step 320) of the at least three aliquots are stained (e.g., fluorescently stained) for the presence of one or more tumor markers (e.g., one or more epithelial markers) (step 321) and optionally counterstained (e.g., with DAPI) (step 322) (see FIG. 4B). This provides for a tumor marker aliquot (see FIG. 1C). In some embodiments, a third aliquot (step 330) of the at least three aliquots are prepared as a negative control (e.g., cells within this third aliquot are incubated with one or more detection reagents, but not stained for the presence of any biomarkers) (step 331). In some embodiments, the third aliquot (a negative control aliquot) is incubated with one or more detection reagents that are used to label the tumor marker and the cell proliferation marker in the first and second aliquots, respectively. In some embodiments, the negative control aliquot is optionally counterstained (e.g., with DAPI) (step 332) (FIG. 4C). Table 1 sets forth an exemplary (but non-limiting) set of aliquots derived from a single representative sample for use in downstream processing.

TABLE 1

| | DAPI | Primary | Secondary | |
|---|---|---|---|---|
| Aliquot | (DNA) | antibody | antibody | Comment |
| Negative | Yes | None | Alexa Fluor | Control for |
| Control | | | 647 | background |
| Aliquot | | | | fluorescence |
| | | | | and non-specific |
| | | | | binding |
| Tumor | Yes | Cytokeratin | Alexa Fluor | Locates tumor |
| Marker | | (CK) | 647 | cell population |
| Aliquot | | | | position on |
| | | | | histogram |
| Cell | Yes | Ki-67 | Alexa Fluor | Marker for |
| Proliferation | | | 647 | proliferating |
| Marker | | | | cells |
| Aliquot | | | | |

In some embodiments, at least a fourth aliquot of the sample is obtained and stained for the presence of yet a different biomarker. In some embodiments, a normal cell marker (e.g., CD3) may be stained to provide a normal marker aliquot to assist with the gating strategy. By way of example, CD3-positive cells have a lower side scatter than CK-positive tumor cells. As described further herein, when the fluorescence of each of these populations is plotted on a scatter plot against the side scatter, a gate may be drawn in between these two populations to define the cutoff for high side scatter cells vs. low side scatter cells.

In some embodiments, cells within a first aliquot (e.g., a proliferation marker aliquot), a second aliquot (e.g., a tumor marker aliquot), and optionally a third aliquot (e.g., a normal marker aliquot) are stained in simplex assays. In a simplex assay, a single detectable moiety is used for all biomarker-specific reagents bound to the sample. Thus, for example, an IHC assay for a single biomarker using a single fluorophore would be considered a “simplex IHC assay.” In some embodiments, the detectable label or reporter moiety used in the simplex assay is a fluorophore.

In other embodiments, cells within the first, second aliquots, and optional third aliquots are stained in multiplex assays. A multiplex assay involves staining multiple biomarkers in a single aliquot where at least some of the biomarkers are differentially labeled. Thus, for example, an IHC assay for two distinct biomarkers in the same sample, with a different fluorophore label for each biomarker would be considered a “multiplex IHC assay.” By way of example, a second aliquot (e.g., a tumor marker aliquot) of the representative sample may be stained for the presence of a tumor cell marker (e.g., a cytokeratin) and a proliferation marker (e.g., Ki-67), where each marker is labeled with a different fluorophore. By way of another example, a second aliquot (e.g., a tumor marker aliquot) of the representative sample may be stained for the presence of a tumor cell marker (e.g., a cytokeratin) and a normal cell marker (e.g., CD3), where each marker is labeled with a different fluorophore.

Exemplary fluorophores include several common chemical classes, such as coumarins, fluoresceins (or fluorescein derivatives and analogs), rhodamines, resorufins, luminophores and cyanines. Additional examples of fluorescent molecules can be found in Molecular Probes Handbook—A Guide to Fluorescent Probes and Labeling Technologies, Molecular Probes, Eugene, OR, ThermoFisher Scientific, 11th Edition. Exemplary fluorescent dyes compatible with mpIHC/mpICC and methodologies of using the same are disclosed at, for example, Gorris, Hofman, and Parra. In other embodiments, the fluorophore is selected from xanthene derivatives, cyanine derivatives, squaraine derivatives, naphthalene derivatives, coumarin derivatives, oxadiazole derivatives, anthracene derivatives, pyrene derivatives, oxazine derivatives, acridine derivatives, arylmethine derivatives, and tetrapyrrole derivatives. In other embodiments, the fluorescent moiety is selected from a CF dye (available from Biotium), DRAQ and CyTRAK probes (available from BioStatus), BODIPY (available from Invitrogen), Alexa Fluor (available from Invitrogen), DyLight Fluor (e.g. DyLight 649) (available from Thermo Scientific, Pierce), Atto and Tracy (available from Sigma Aldrich), FluoProbes (available from Interchim), Abberior Dyes (available from Abberior), DY and MegaStokes Dyes (available from Dyomics), Sulfo Cy dyes (available from Cyandye), HiLyte Fluor (available from AnaSpec), Seta, SeTau and Square Dyes (available from SETA BioMedicals), Quasar and Cal Fluor dyes (available from Biosearch Technologies), SureLight Dyes (available from APC, RPEPerCP, Phycobilisomes) (Columbia Biosciences), and APC, APCXL, RPE, BPE (available from Phyco-Biotech, Greensea, Prozyme, Flogen). Methods of staining samples are described in United States Patent Publication Nos. 2023/019258, 2019/0204330, 2017/0089911, and 2019/0187130; in U.S. Pat. Nos. 5,583,001, 10,168,336, and 10,041,950; and in PCT Publication No. WO/2017/085307, the disclosures of which are hereby incorporated by reference herein in their entireties.

### Cell Proliferation Marker

In some embodiments, cells within any aliquot may be stained for the presence of one or more cell proliferation markers. Cellular proliferation is the most fundamental process in living organisms, and as such is precisely regulated by the expression level of proliferation-associated genes. Loss of proliferation control is a hallmark of cancer, and it is thus not surprising that growth-regulating genes are abnormally expressed in tumors relative to the neighboring normal tissue. Proliferative changes may accompany other changes in cellular properties, such as invasion and ability to metastasize, and therefore could affect patient outcome. As used herein, the term “cell proliferation marker” refers to any marker molecule known in the art to be characteristic for the proliferation status of cells. The proliferation status may be, e.g., a status of actively proliferating cells, of retarded cell proliferation, of arrested cell proliferation, of senescent cells, of terminally differentiated cells, of apoptosis, etc. In some embodiments, the cell proliferation marker is a marker molecule characteristic for active cell proliferation. In other embodiments, the cell proliferation marker may be a molecule characteristic for arrested, terminally differentiated, senescent, or apoptotic cells.

In some embodiments, cell proliferation markers include genes engaged in DNA replication, such as e.g., proteins of the pre-initiation complex or of the replication fork. Such molecules may comprise, e.g., helicases, such as eucaryotic helicase or MCM proteins (MCM2, MCM3, MCM4, MOMS, MCM6, MCM7), protein TP as disclosed in PCT Publication Nos. WO/0050451 and WO/0217947 (the disclosures of which are hereby incorporated by reference herein in their entireties) kinases or phosphatases engaged in the replication process such as, e.g., CDC6, CDC7 protein kinase, Dbf4, CDC14 protein phosphatase, CDC45 and MCM10. In other embodiments, cell proliferation markers may comprise proteins engaged in the processive replication fork such as, e.g., PCNA or DNA polymerase delta, replication protein A (RPA), replication factor C (RFC), and FEN1. In yet other embodiments, the cell proliferation markers may comprise molecules necessary for the maintenance of cell proliferation such as Ki-67, Ki-S5, or Ki-S2. In further embodiments, the cell proliferation markers are characteristic of retarded or ceased cell proliferation such as, e.g., a senescence marker, a cell cycle arrest marker, a marker characteristic for terminally differentiated cells, or an apoptosis marker. Non-limiting examples of such cell proliferation markers include p21, p27, Caspases, BAD, CD95, fas-ligand, parp-proteins, etc.

Non-limiting examples of antibodies against the Ki-67 cell proliferation marker (anti-Ki-67 antibodies) include those belonging to the MIB@-family, such as MIB-1, MIB-2, MIB-5, MIB-7, MIB-21, and MIB-24.

Other non-limiting examples of antibodies specific to proliferation markers are set forth in Table 2:

TABLE 2

| | Specific | | |
|---|---|---|---|
| Marker Type | protein detected | Name of clone | Vendor |
| Proliferation | Ki-67 | 30-9 | Roche |
| Proliferation | Ki-67 | Ki-67 | BioLegend |
| Proliferation | Ki-67 | D3B5 | Cell Signaling |
| | | | Technology |
| Proliferation | Ki-67 | 20Raj1 | eBioscience |
| Proliferation | p16 | E6H4 | Roche |
| Proliferation | p21 | 12D1 | Cell Signaling |
| | | | Technology |
| Proliferation | p27 | Y236 | Abcam |
| Proliferation | Cyclin A1 + A2 | EPR18054 | Abcam |
| Proliferation | Cyclin D1 | SP4-R | Roche |
| Proliferation | Cyclin D1 | EPR2241 | Abcam |
| Proliferation | Cyclin D1 | 92G2 | Cell Signaling |
| | | | Technology |
| Proliferation | Cyclin E | EP435E | Abcam |
| Proliferation | Cyclin E | HE12 | Thermo Fisher |
| Proliferation | CDT1 | EPR17891 | Abcam |
| Proliferation | CDT1 | D10F11 | Cell Signaling |
| | | | Technology |
| Proliferation | Geminin | EPR14637 | Abcam |
| Proliferation | Geminin | F7 | Santa Cruz |
| Proliferation | pRB | D20B12 | Cell Signaling |
| | | | Technology |
| Proliferation | pHH3 | D2C8 | Cell Signaling |
| | | | Technology |
| Proliferation | PCNA | PC10 | Abcam |
| Proliferation | PCNA | PC10 | Cell Signaling |
| | | | Technology |
| Proliferation | Cyclin B1 | Y106 | Abcam |
| Proliferation | MCM2 | EPR4120 | Abcam |
| Proliferation | MCM2 | E8 | Santa Cruz |

### Tumor Markers

In some embodiments, cells within any aliquot are stained for the presence of one or more tumor markers. Examples of tumor markers are set forth in Table 3:

TABLE 3

| | Specific | | |
|---|---|---|---|
| Marker Type | protein detected | Name of clone | Vendor |
| Tumor | Cytokeratin 8/18 | B22.1 & B23.1 | Roche |
| Tumor | Pan-cytokeratin | AE3 | Roche |
| Tumor | Pan-cytokeratin | AE1/AE3/PCK26 | Roche |
| Tumor | E-Cadherin | 24E10 | Cell Signaling |
| | | | Technology |
| Tumor | Ep-CAM | Ber-EP4 | Roche |
| Tumor | CK8 | EP1628Y | Abcam |
| Tumor | ER | EPR4097 | Abcam |
| Tumor | ER | D6R2W | Cell Signaling |
| | | | Technology |
| Tumor | Pan-Cytokeratin | ae183 | eBioscience |
| Tumor | Pan-Cytokeratin | A1/A3 | eBioscience |
| Tumor | S100 | 4C4.9 | Abcam |
| Tumor | MITF | EPR26363-10 | Abcam |
| Tumor | Melan-A | EPR20380 | Abcam |
| Tumor | Pan-Cytokeratin | C11 | Cell Signaling |
| | | | Technology |

In some embodiments, the one or more tumor markers includes marks of epithelial origin (Cytokeratins, EPCAM, EMA, etc.) or specific markers for cancers of mesynchymal origin, such as S100, Melan-A, MITF, HMB-45, Tyrosinase, PMEL, CSPG4, SM5-1m. In some embodiments, cytokeratin is selected from the group consisting of high molecular weight cytokeratins and/or low molecular weight cytokeratins. In some embodiments, the cytokeratin is selected from the group consisting of CK1, CK2, CK3, CK4, CK5, CK6, CK7, CK8, and CK9. In some embodiments, the cytokeratin is selected from the group consisting of CK10, CK12, CK13, CK14, CK16, CK17, CK18, CK19, and CK20.

In some embodiments, the tumor marker is an epithelial marker. The epithelial markers may be proteins or fragments thereof that are released by the cells by shedding, secretion, or other mechanisms. The term “epithelial marker” as used herein is broadly defined as any one of a wide variety of proteins peptides, polypeptides, group of peptides or proteins, nucleic acids, and related molecules of which the presence or levels of are used to assess the presence of epithelial cells. In some embodiments, the epithelial marker is used to assess the presence of disseminated epithelial cells found in tissues not normally associated with epithelial cells, such as in mesenchymal tissues (e.g., blood, bone marrow), to assess the status of tumor progression (in particular, early detection of metastasis) in cancer patients.

In some embodiments, the epithelial markers may be used to identify disseminated tumor cells derived from solid tumors. Examples of solid tumors that may be identified by the epithelial markers include, but are not limited to the following: carcinoma, adenoma, hepatocellular carcinoma, hepatoblastoma, esophageal carcinoma, thyroid carcinoma, ganglioblastoma, synovioma, Ewing's tumor, colon carcinoma, pancreatic cancer, breast cancer, ovarian cancer, prostate cancer, squamous cell carcinoma, basal cell carcinoma, adenocarcinoma, renal cell carcinoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, craniopharyngioma, ependymoma, pinealoma, retinoblastoma, rectal carcinoma, cancer of the thyroid, head and neck cancer, and cancer of the endometrium.

Yet other examples tumor markers may include, but are not limited to, cytokeratins detectable with the pan keratin antibody (e.g., basic cytokeratins, many of the acidic cytokeratins), other cytokeratins such as cytokeratin 7 (CK7) and cytokeratin 20 (CK20), chromogranin, synaptophysin, CD56, thyroid transcription factor-1 (TTF-1), p53, leukocyte common antigen (LCA), vimentin, smooth muscle actin, or the like (e.g., see Capelozzi, V., J Bras Pneumol. 2009; 35(4):375-382, the disclosure of which is hereby incorporated by reference herein in its entirety). The tumor markers are not limited to proteins detectable with IHC; for example, the tumor cell-specific biomarker may be a nucleic acid sequence of interest detectable with ISH techniques.

### Normal Cell Markers

In some embodiments, cells within any aliquot are stained for the presence of one or more normal cell markers. Examples of suitable normal cell markers include, but are not limited, to CD3 and CD45, and CD8. It is ideal to use markers that will stain small normal cells, like lymphocytes, which will have a low side scatter compared to tumor cells, and cells that are found in appreciable amounts in the tumor microenvironment.

Other non-limiting examples of antibodies specific to normal cell markers are set forth in Table 4:

TABLE 4

| | Specific | | |
|---|---|---|---|
| Marker Type | protein detected | Name of clone | Vendor |
| Normal | CD3 | 2GV6 | Roche |
| Normal | CD3 | CD3-12 | Abcam |
| Normal | CD8 | SP57 | Roche |
| Normal | CD8a | AMC908 | eBioscience |
| Normal | CD45 | 2B11 & PD7/26 | Roche |
| Normal | CD45 | HI30 | BioLegend |
| Normal | CD45 | 30-F11 | BioLegend |
| Normal | CD45 | PD7/26 | eBioscience |
| Normal | CD45 | 2D1 | R&D |
| Normal | aSMA | EPR5368 | Abcam |
| Normal | aSMA | D4K9N | Cell Signaling |
| | | | Technology |
| Normal | aSMA | 1A4 | eBioscience |
| Normal | CD163 | EPR14643-36 | Abcam |
| Normal | CD20 | L26 | eBioscience |
| Normal | CD20 | L26 | Roche |
| Normal | CD31 | JC70 | Roche |
| Normal | CD31 | JC/70A | Abcam |
| Normal | CD31 | EPR3094 | Abcam |
| Normal | vimentin | D21H3 | Cell Signaling |
| | | | Technology |
| Normal | vimentin | V9 | Roche |
| Normal | vimentin | EP21 | Cell Marque |

### Counterstain

In some embodiments, the cells within any aliquot may be counterstained to identify DNA. In some embodiments, the cells are counterstained with Hoescht, 7-AAD, Propidium Iodide, DRAQ5, DRAQ7, Cytophase Violet, Helix NP, https://www.biolegend.com/en-us/cell-cycle/dna-dyes. In some embodiments, the cells within any aliquot are stained with 4′,6-diamidino-2-phenylindole (DAPI).

### Cytometric Analysis of the Prepared Aliquots

The present disclosure also provides methods of obtaining cytometry data for each prepared aliquot derived from the representative sample and evaluating the obtained flow cytometry data (see FIG. 1A, steps 30 and 40; see also FIGS. 1B and 1C). In some embodiments, the one or more aliquots prepared for cytometric analysis are evaluated using cytometry data such that different cell types may be identified (e.g., normal cells, tumor cells, normal cells staining positive for a cell proliferation marker, tumor cells staining positive for a cell proliferation marker, etc.). In some embodiments, the evaluation comprises quantifying the percentages of tumor cells staining positive for a cell proliferation marker and normal cells staining positive for the cell proliferation marker.

Various methods of cytometrically assaying-stained cells in an aliquot derived from a representative sample include flow cytometrically assaying using a flow cytometer, cell cytometrically assaying a labeled cell suspension, e.g., by using a cell cytometer, and the like. In some embodiments, additional cellular parameters, assayed cytometrically, may also find use in detecting neoplastic cells of the present disclosure. Accordingly, various methods of cytometrically assaying a labeled cell suspension to measure various cellular parameters may be employed.

Flow cytometry is a methodology using multi-parameter data for identifying and distinguishing between different particle (e.g., cell) types i.e., particles that vary from one another in terms of label (wavelength, intensity), size, etc., in a fluid medium. In flow cytometrically analyzing a sample, an aliquot of the sample is first introduced into the flow path of the flow cytometer. When in the flow path, the cells in the sample are passed substantially one at a time through one or more sensing regions, where each of the cells is exposed separately and individually to a source of light at a single wavelength (or in some instances two or more distinct sources of light) and measurements of cellular parameters, e.g., light scatter parameters, and/or marker parameters, e.g., fluorescent emissions, as desired, are separately recorded for each cell. The data recorded for each cell is analyzed in real time or stored in a data storage and analysis means, such as a computer, for later analysis, as desired. In flow cytometry-based methods, the cells are passed, in suspension, substantially one at a time in a flow path through one or more sensing regions where in each region each cell is illuminated by an energy source. The energy source may include an illuminator that emits light of a single wavelength, such as that provided by a laser (e.g., He/Ne or argon) or a mercury arc lamp or an LED with appropriate filters. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 405 nm, 535 nm, 561 nm, 635 nm, 642 nm, and the like. Following excitation of a labeled specific binding member (e.g., a primary antibody) bound to a polypeptide by an energy source, the excited label emits fluorescence and the quantitative level of the polypeptide on each cell may be detected, by one or more fluorescence detectors, as it passes through the one or more sensing regions.

The flow cytometry data generated can be plotted into scatter plots and/or histograms and divided into regions. Regions are shapes that are drawn or positioned (either manually or automatically) around a population of interest on a one or two parameter scatter plot or histogram. Exemplary region shapes include two dimensional polygons, circles, ellipses, irregular shapes, or the like. When a region is used to limit or isolate cells or events that are drawn or positioned on a scatter plot or histogram, such that those isolated cells or events can be manifested in a subsequent scatter plot or histogram, this process is referred to as gating.

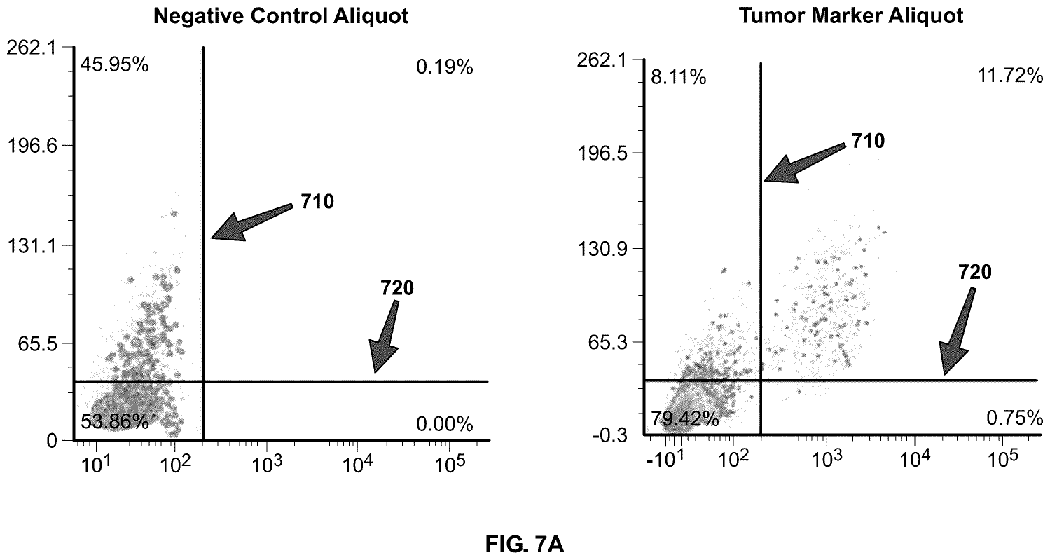

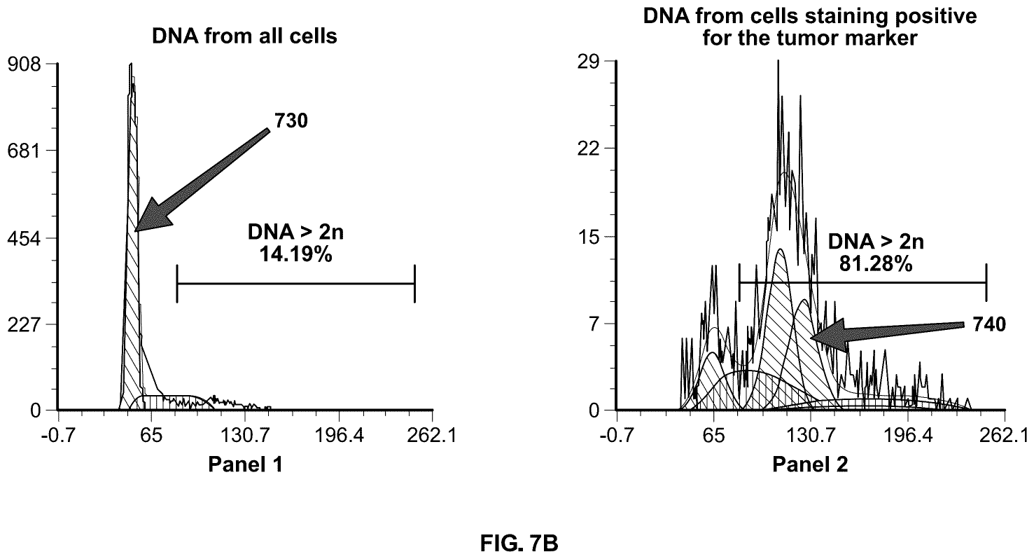

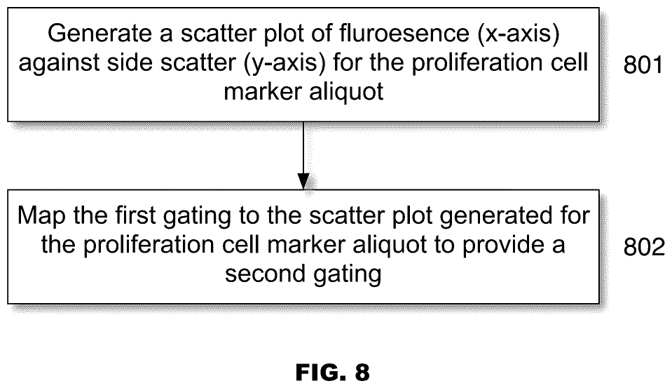

To select an appropriate gate, the data is plotted to obtain appropriate separation of subpopulations of particles, e.g., by adjusting the configuration of the instrument, including e.g., excitation parameters, collection parameters, compensation parameters, etc. In some instances, this procedure is completed by plotting forward scatter content (FSC) versus side scatter content (SSC) on a two-dimensional dot plot. Alternatively, fluorescence intensity may be plotted versus SSC (see, e.g., FIGS. 7A, 9A, 10A, and 11A, herein). The flow cytometry operator then selects the desired subpopulation of cells (i.e., those cells within the gate) and excludes cells which are not within the gate. Where desired, the operator may select the gate by drawing one or more lines (e.g., vertical and/or horizontal lines) around the desired subpopulation using a cursor on a computer screen. Only those cells within the gate are then further analyzed by plotting the other parameters for these cells, such as fluorescence. The process of gating does not change the data, i.e., it only depicts it in a way that flow cytometry analysts find themselves familiar with.

Non-limiting examples of suitable flow cytometer systems include those available from commercial suppliers including but not limited to, e.g., Becton-Dickenson (Franklin Lakes, NJ), Life Technologies (Grand Island, NY), Acca Biosciences (San Diego, CA), Beckman-Coulter, Inc. (Indianapolis, IN), Bio-Rad Laboratories, Inc. (Hercules, CA), Cytonome, Inc. (Boston, MA), Amnis Corporation (Seattle, WA), EMD Millipore (Billerica, MA), Sony Biotechnology, Inc. (San Jose, CA), Stratedigm Corporation (San Jose, CA), Union Biometrica, Inc. (Holliston, MA), Cytek Development (Fremont, CA), Propel Labs, Inc. (Fort Collins, CO), Orflow Technologies (Ketchum, ID), handyem inc. (Quebec, Canada), Sysmex Corporation (Kobe, Japan), Partec Japan, Inc. (Tsuchiura, Japan), Bay bioscience (Kobe, Japan), Furukawa Electric Co. Ltd. (Tokyo, Japan), On-chip Biotechnologies Co., Ltd (Tokyo, Japan), Apogee Flow Systems Ltd. (Hertfordshire, United Kingdom), and the like.

In some embodiments, flow cytometry data is obtained for each prepared aliquot (see steps 30 and 130 of FIGS. 1A and 1B). In some embodiments, flow cytometry data is obtained for each of at least two aliquots derived from the representative sample. In other embodiments, flow cytometry data is obtained for each of at least three aliquots derived from the representative sample. In yet other embodiments, flow cytometry data is obtained for each of at least four aliquots derived from the representative sample. In some embodiments, the flow cytometry data from each aliquot derived from the representative sample comprises a scatter plot of fluorescence intensity versus side scatter content (SSC). Examples of scatter plots are illustrated in FIGS. 7A,9A, 10A, 10C, 11A, 11C, and 12A where each scatter plot includes fluorescence intensity plotted on the x-axis and side scatter content is plotted on the y-axis.

In some embodiments, at least two gatings, such as two sequential gatings, are performed on the obtained flow cytometry data to identify cell proliferation marker positive normal cells and cell proliferation marker positive tumor cells (see, e.g., FIG. 1C). With reference to FIG. 5, in some embodiments, a first gating is conducted to identify cells that stain positive for a tumor cell marker (e.g., a cytokeratin) (step 500). In some embodiments, the first gating is optionally confirmed by comparing DNA from all cells as compared with cells within the first gate (step 510). Subsequently, a second gating is conducted to identify cell proliferation marker positive normal cells and cell proliferation marker positive tumor cells in the aliquot stained for the presence of the one or more proliferation markers (step 520). In some embodiments, the second gating is optionally confirmed by comparing the DNA from all cells staining positive for the proliferation marker to DNA of normal cells and tumor cells (step 530).

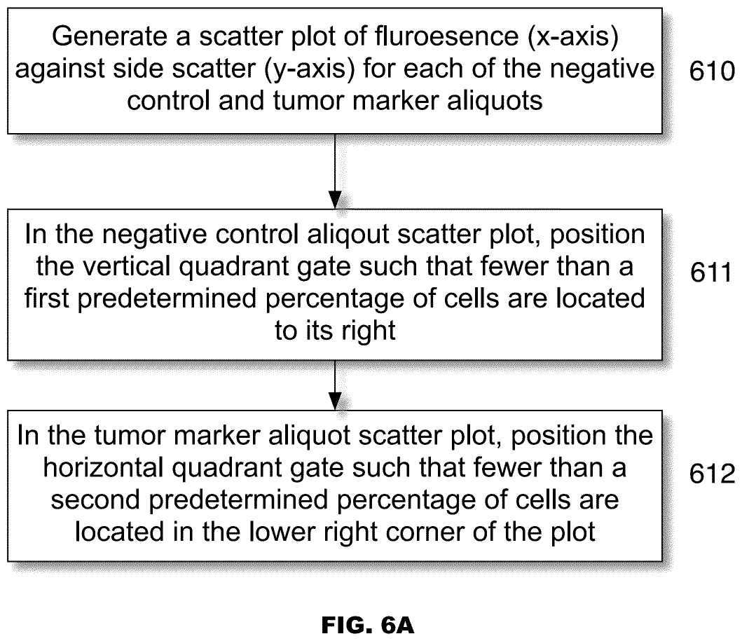

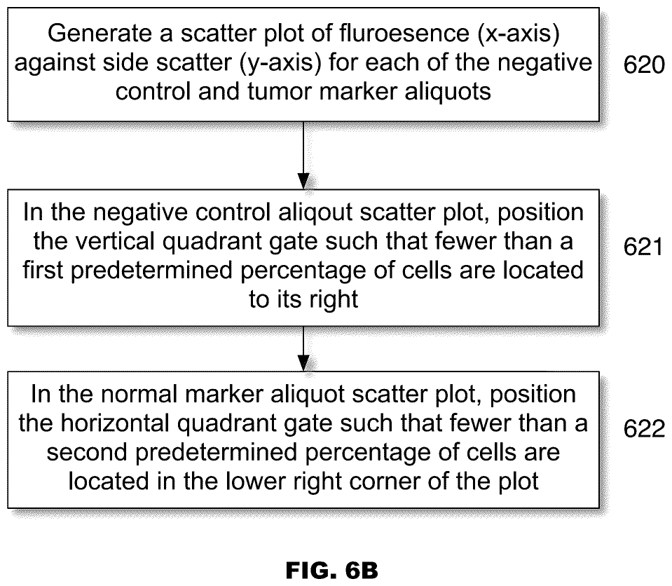

In some embodiments, the first gating is performed on flow cytometry data derived from a negative control aliquot, an aliquot stained for the presence of a tumor marker, and optionally an aliquot stained for the presence of a normal cell marker. With reference to FIGS. 6A and 6B, scatter plots of fluorescence intensity versus side scatter content for each of a negative control aliquot (e.g., an aliquot incubated with one or more detection reagents and optionally counterstained with DAPI), a tumor marker aliquot (e.g., an aliquot stained for the presence of a tumor marker, such as a cytokeratin, and optionally counterstained), and optionally a normal marker aliquot (e.g., an aliquot stained for the presence of a normal cell marker) are generated from obtained flow cytometry data (step 610). Next, a vertical quadrant gate is placed. This vertical quadrant gate is to identify where the fluorescent signal corresponds to background signals. The higher this number is, the more non-specific fluorescent signal is being counted as real cells.

In some embodiments, a vertical quadrant gate is placed in the negative control aliquot scatter plot at or near the right edge of the negative control cells. In other embodiments, a vertical quadrant gate is placed in the negative control aliquot scatter plot such that less than a predetermined percentage of cells are located to the right of the vertical quadrant gate. In some embodiments, the predetermined percentage is about 10%, about 9%, about 8%, about 7%, about 6%, about 5%, about 4%, about 3%, about 2.5%, about 2%, about 1.5%, about 1.25%, about 1%, about 0.75%, about 0.5%, about 0.25% etc. (step 611). In some embodiments, a vertical quadrant gate is placed within the negative control aliquot scatter plot such that fewer than about 1% of the cells are located to the right of the vertical quadrant gate.

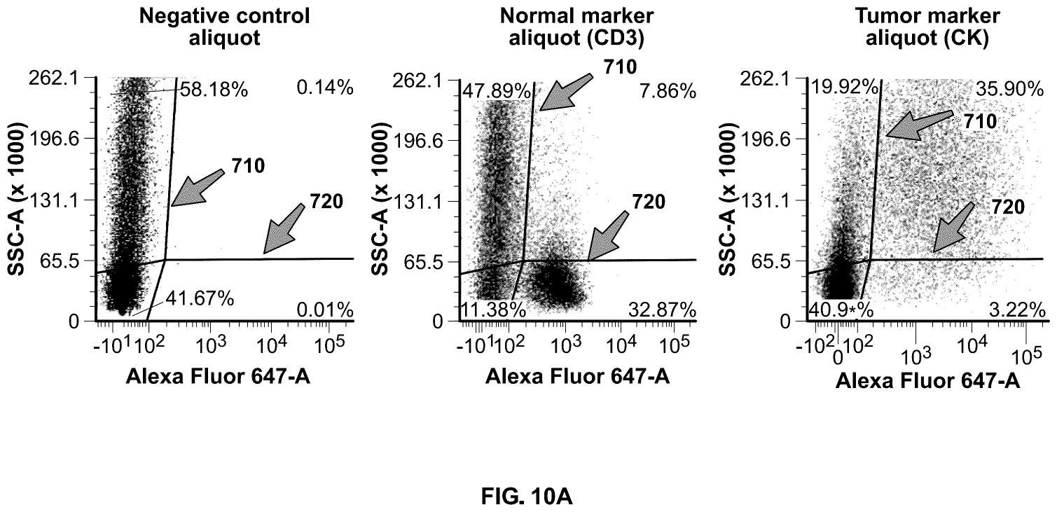

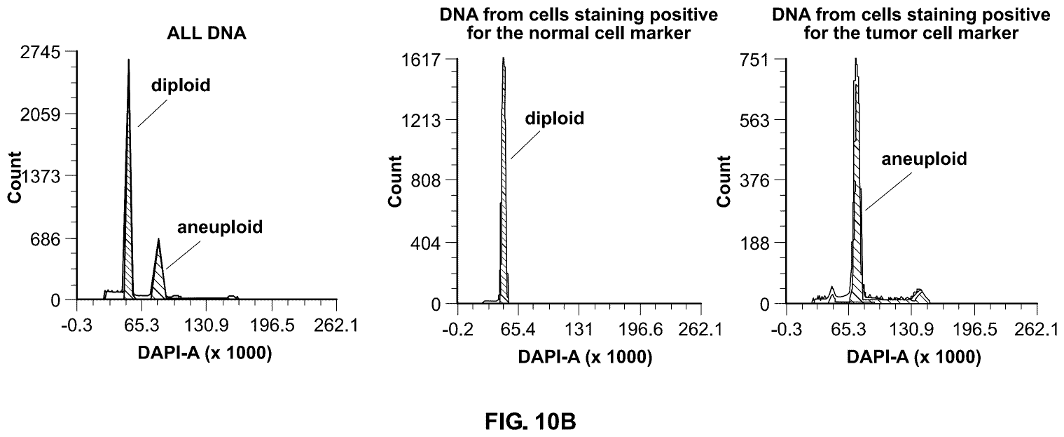

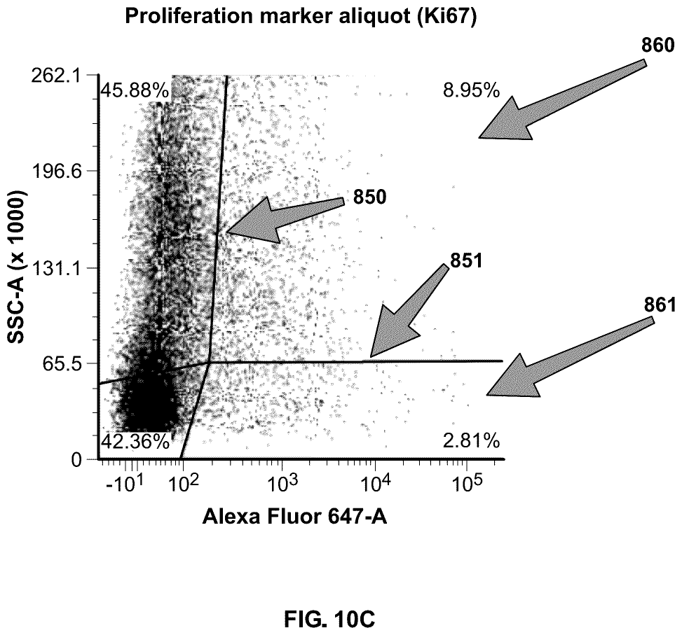

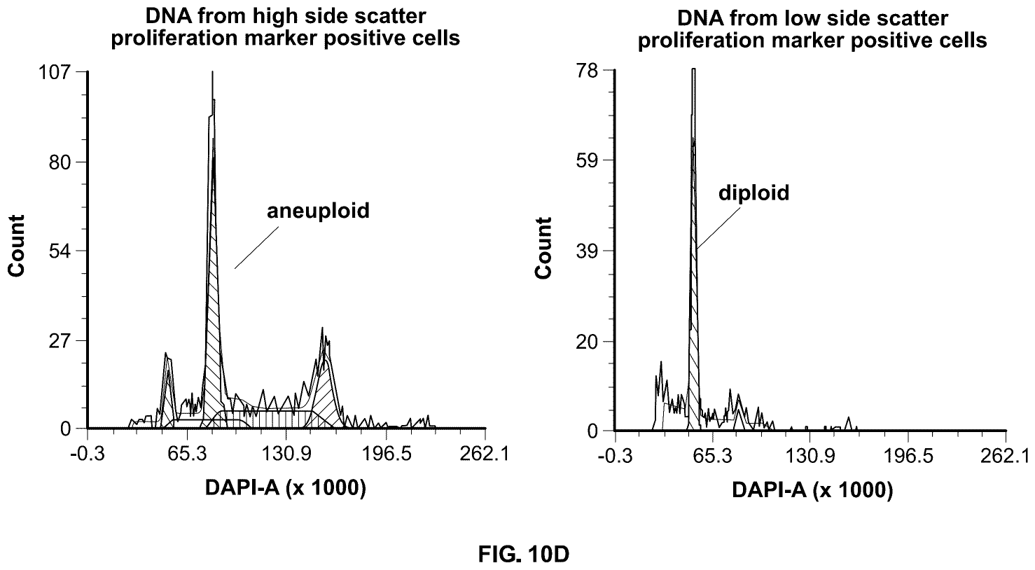

In some embodiments, the vertical quadrant gate placed on the negative control aliquot scatter plot is transcribed to the same position on the tumor marker aliquot scatter plot the optional normal marker aliquot scatter plot (compare the two histograms present in FIG. 7A; also compare the three histograms present in FIG. 10A). For instance, FIG. 7A illustrates the placement of the vertical quadrant gate 710 in the negative control aliquot scatter plot and its transcription to the same location (fluorescence intensity) in the tumor marker aliquot scatter plot. Likewise, FIG. 10A depicts the placement of the vertical quadrant gate 710 in the negative control aliquot scatter plot and its transcription to the same location (fluorescence intensity) in the tumor marker aliquot scatter plot and in the normal marker aliquot scatter plot. It is believed that cells that stain positive for the tumor marker in the tumor marker aliquot scatter plot fall to the right of the vertical quadrant gate.

The first gating further comprises the step of placing the horizontal quadrant gate (steps 612 and 622). In some embodiments, in the tumor marker aliquot scatter plot, a horizontal quadrant gate is placed such that fewer than a predetermined percentage of cells are in the lower right of the tumor marker aliquot scatter plot. In some embodiments, the predetermined percentage is about 10%, about 9%, about 8%, about 7%, about 6%, about 5%, about 4%, about 3%, about 2.5%, about 2%, about 1.5%, about 1.25%, about 1%, about 0.75%, about 0.5%, about 0.25% etc. In other embodiments, a horizontal quadrant gate is placed in the tumor marker aliquot scatter plot such that fewer than about 1% of the cells are in the lower right of the tumor marker aliquot scatter plot. In some embodiments, the horizontal quadrant gate placed on the tumor marker aliquot scatter plot is transcribed to the same position on the negative control aliquot scatter plot (see the two panels of FIG. 7A). FIG. 7A illustrates the placement of the horizontal quadrant gate 720 in the tumor marker aliquot scatter plot and its transcription to the same location (fluorescence intensity) in the negative control aliquot scatter plot. It is believed that cells that stain positive for the tumor marker should fall into the top right of the quadrant.

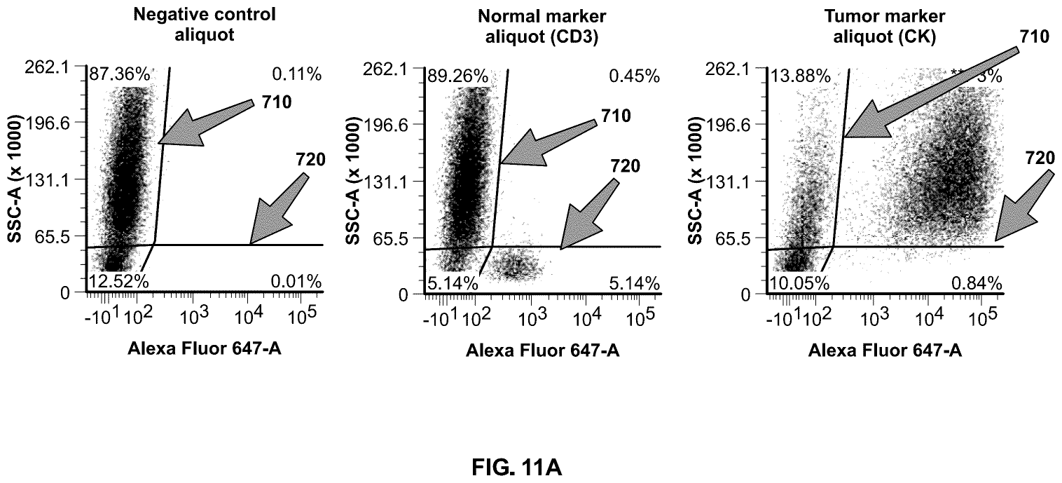

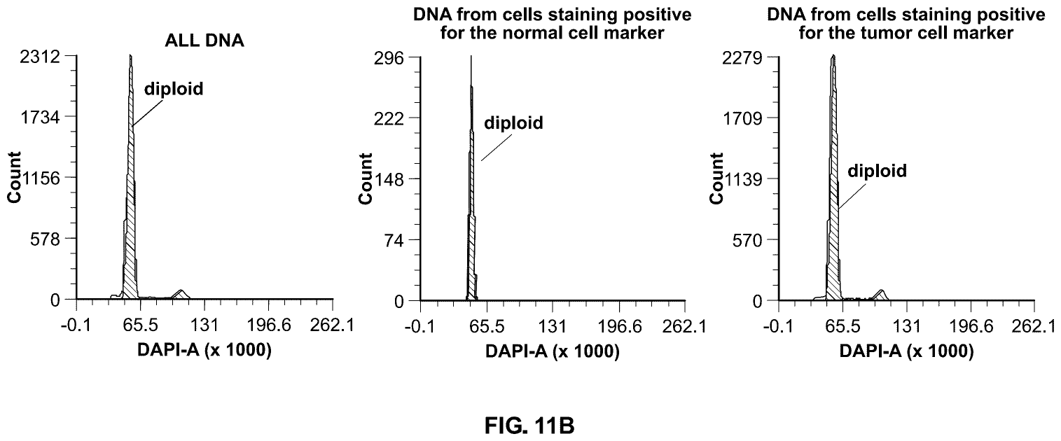

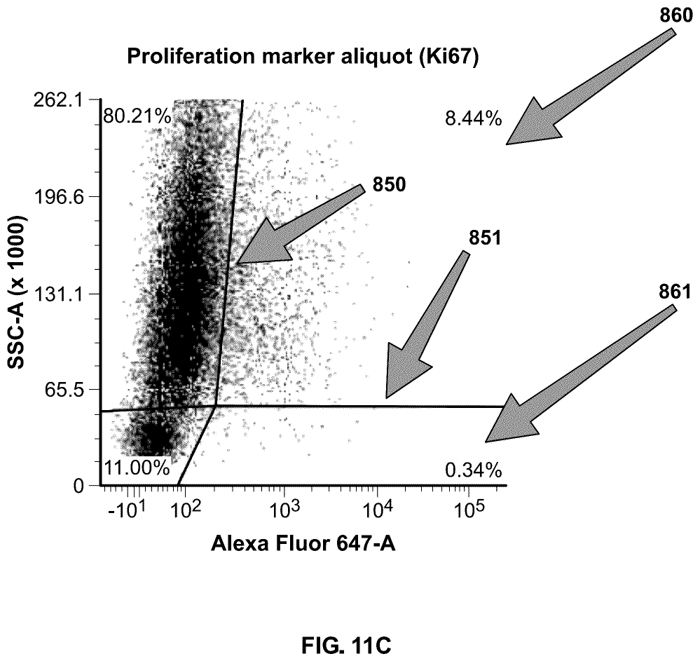

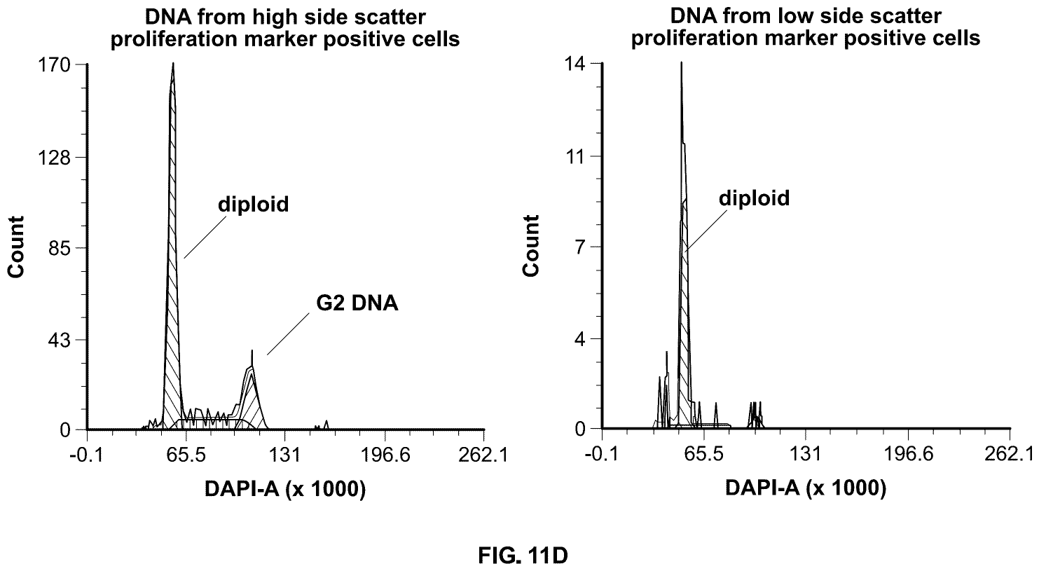

In some embodiments, the first gating is performed using flow cytometry data obtained from an aliquot stained for the presence of a normal cell marker (e.g., CD3). For instance, in some embodiments a horizontal quadrant gate is placed in the normal marker aliquot scatter plot such that fewer than a predetermined percentage of cells are in the lower right of the normal marker aliquot scatter plot. As further illustrated in FIGS. 10A and 11A, the horizontal marker is placed so that most normal marker (CD3) positive cells are in the lower right quadrant, and most tumor marker (CK) positive cells are in the upper right quadrant. In some embodiments, the predetermined percentage is about 10%, about 9%, about 8%, about 7%, about 6%, about 5%, about 4%, about 3%, about 2.5%, about 2%, about 1.5%, about 1.25%, about 1%, about 0.75%, about 0.5%, about 0.25% etc. In some embodiments, a horizontal quadrant gate is placed in the normal marker aliquot scatter plot such that fewer than about 1% of the cells are in the lower right of the normal marker aliquot scatter plot. In some embodiments, the horizontal quadrant gate placed on the normal marker aliquot scatter plot is transcribed to the same position on the negative control aliquot scatter plot and the tumor marker aliquot scatter plot (see the three panels of FIGS. 10A and 11A).

In some embodiments, the first gating may be optionally confirmed by plotting DNA content as a histogram. DNA from cells staining positive for the tumor marker (upper right quadrant of the tumor marker aliquot scatter plot, see FIGS. 7A, 10A, and 11A) often have a high DNA content relative to DNA from all cells. Said another way, tumor cells often have a much higher percentage of cells with a high DNA content. The reason tumor cells often contain high DNA content is that tumor cells are often aneuploid; and they often have amplifications of segments of chromosomes, or in some cases, whole genome duplications. For aneuploid tumors, concurrent analysis of DNA content (as shown in FIGS. 7B, 10B, and 11B) can confirm placement of gates by serving as an additional “tumor marker.”

In some embodiments, the histogram plot diagrams provide data density for a given parameter. In the examples shown in FIGS. 7B, 10B, and 11B, the histogram plot diagram provides along an x-axis the parameter of interest (here DNA content); and provides the count for each parameter along the y-axis. In some embodiments, a line can be drawn, connecting the counts at some or all the parameters. In some embodiments, the peaks and valleys provide information about the relative density of events for given parameters. Thus, the histogram plot diagram provides a visual representation of the density of the data, that is, how many occurrences of an event happened at a particular parameter.

In some embodiments, the DNA from all cells (730) may be examined (see FIG. 7B, panel 1) and compared to the DNA from the tumor cells (740), i.e., the cells staining positive for the tumor marker (see FIG. 7B, panel 2). With reference to FIG. 7B, the horizontal bars running parallel to the x-axis in panels 1 and 2 indicate regions where one would expect to find high DNA content. The high DNA content found in Panel 2 confirms that accurate horizontal and vertical gates were placed in FIG. 7A.

Likewise, FIGS. 10B and 11B compare DNA histograms from a negative control aliquot (all cells) to DNA histograms from a normal marker aliquot (CD3 positive cells) and a tumor marker aliquot (CK positive cells). The DNA histogram from the negative control aliquot shows two DNA peaks, one corresponding to diploid DNA and the other corresponding to aneuploid DNA. In CD3+ cells, only the diploid peak is observed, and in CK+ cells only the aneuploid peak is observed, thus confirming that accurate horizontal and vertical gates were placed in FIGS. 10A and 11A, respectively (thus confirming the accuracy of the first gating operation).

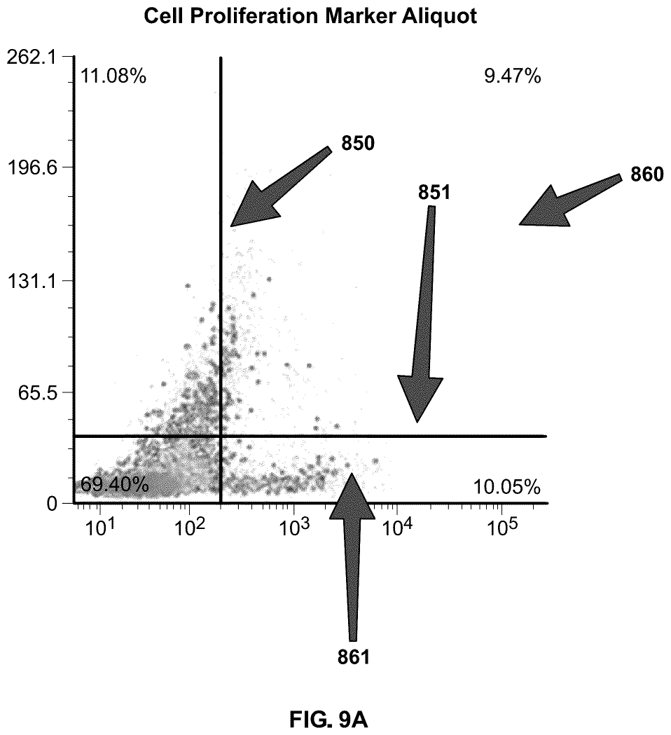

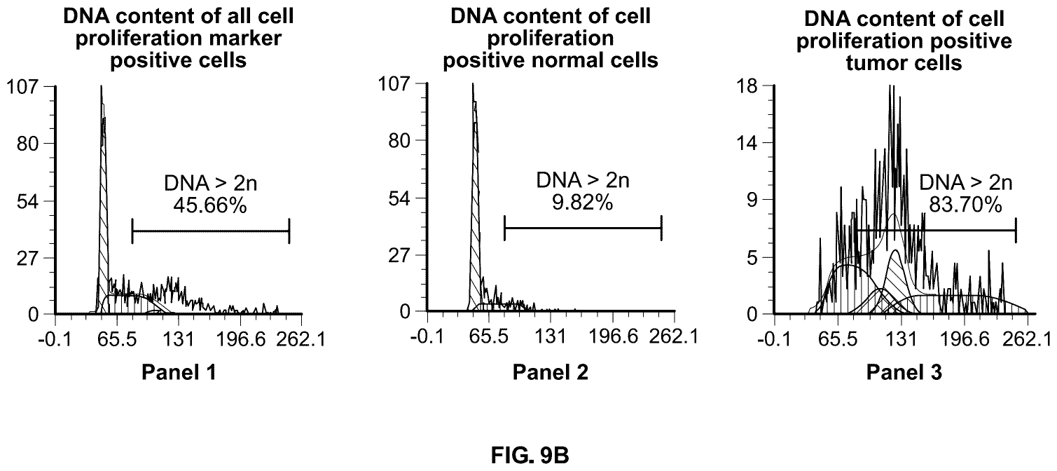

In some embodiments, the second gating is performed by mapping the first gating (horizontal and vertical quadrant gates) to the flow cytometry data derived from the aliquot stained for the presence of the proliferation marker (step 520). More specifically, in some embodiments, a scatter plot of fluorescence (x-axis) is plotted against side scatter content (y-axis) for the proliferation marker aliquot (FIG. 8, step 801). In some embodiments, the first gating is mapped to the proliferation marker aliquot scatter plot to provide the second gating (step 802). In some embodiments, the identical gating positions from the first gating operating are mapped to the proliferation marker aliquot scatter plot. For instance, the vertical and horizontal quadrant gates 710 and 720, respectively, from FIG. 7A are mapped from the negative control aliquot scatter plot, and the tumor marker aliquot scatter plot to the identical vertical and horizontal positions (850 and 851, respectively, of FIGS. 9A, 10C, and 11C) in the cell proliferation marker aliquot scatter plot. Likewise, the vertical and horizontal quadrant gates 710 and 720, respectively, from FIGS. 10A and 11B are mapped from the negative control aliquot scatter plot, the tumor marker aliquot scatter plot, and the optional normal marker aliquot scatter plot to the identical vertical and horizontal positions (850 and 851, respectively, of FIGS. 9A, 10C, and 11C) in the cell proliferation marker aliquot scatter plot.

As noted herein, the second gating may be optionally confirmed by plotting DNA content as a histogram (step 530). For example, and with reference to FIG. 9B, the DNA from all cell proliferation marker positive cells (Panel 1) may be compared to the proliferation marker positive normal cells (Panel 2) and proliferation marker positive tumor cells (Panel 3). The horizontal bars running parallel of the x-axis in each of the panels of FIG. 9B indicate high DNA content. It is believed that DNA from Ki-67 positive tumor cells should have a high DNA content relative to DNA from normal Ki-67 positive cells. With reference to FIGS. 10D and 11D, DNA content from high side scatter proliferation marker positive cells appear aneuploid like the tumor marker positive population; while DNA content from low side scatter proliferation marker positive cells appear diploid like the normal marker positive cell population, confirming the optimal placement of the gates.

Finally, the percentage of cell proliferation marker positive normal cells (quadrant 861) and the percentage cell proliferation marker positive tumor cells (quadrant 860) is assessed (step 150) (see FIGS. 9A, 10C, and 11C).

### EXAMPLES

### Example 1

### Materials List

IKA tube mill (#0004180001) and disposable grinding chamber (#0004425000), IKA works
CC1 buffer, Ventana Medical Systems, #950-300
Pluriselect filters, Pluriselect USA (#43-50020-03, #43-50040-51, #43-50100-51, #41-50000-03)
Blocking buffer, Ventana Medical Systems, #90103
Cytokeratin 8/18 antibody dispenser, Ventana Medical Systems, clones B22.1 & B 23.1, #760-4344
CD3 antibody dispenser, Ventana Medical Systems, clone 2GV6, #790-4341
Ki-67 antibody dispenser, Ventana Medical Systems, clone 30-9, #790-4286
Phosphate buffered saline, Thermo Fisher Scientific, #10010023
Tween 20, Sigma, #P1379
Bovine Serum Albumin, Sigma, #A2153
Secondary antibodies, Abcam, Goat Anti-Rabbit IgG H&L (Alexa Fluor® 647) preadsorbed #ab150083, Goat Anti-Mouse IgG H&L (Alexa Fluor® 647) preadsorbed #ab150119
DAPI, Sigma, #D9542

### Method

From cancer patients experiencing a surgical resection, leftover fixed tissue was obtained with patient consent after samples were taken for routine diagnostic purposes. From this leftover tissue, tumor tissue was dissected and homogenized to obtain a representative fixed tissue sample. An about 0.5 mg aliquot of this tissue was dissociated to single cells by first incubating at 85° C. in 7.5 ml CC1 buffer for 30 min, and then further homogenizing in an IKA tube mill homogenizer. Following this, any non-dissociated tissue fibers were removed using a series of Pluriselect filters, starting with a 100-micron filter, followed by a 40-micron filter, and then a 20-micron filter. Following filtration, the resulting cells were collected by centrifugation at 1000×g for 5 min, and then exchanged into blocking buffer for 10 min. Cells were distributed into 1.5 mL Eppendorf tubes (˜10\hat{ }7 cells per tube) and collected by centrifugation at 500×g for 1 min. Cells were incubated in 0.3 mL of the primary antibody staining the marker of choice directly from antibody dispensers at existing dilutions for 5 hours to overnight at 4° C., then washed 3×0.5 ml with Phosphate buffered saline containing 0.1% tween 20 and 0.1% bovine serum albumin (wash buffer). Following the final wash, cells were exchanged into 0.3 mL of the appropriately labeled secondary antibody (Goat-anti-mouse or Goat-anti-rabbit labeled with Alexa Fluor 647 at 1:1000) and DAPI (1:1000) for 30 min at 4° C. The labeled cells were washed 2×0.5 mL with wash buffer and then filtered through a 40-micron filter top into a round bottom polystyrene tube for analysis by flow cytometry. Cells were analyzed on a BD FACS Melody equipped with a BRV laser/filter setup or BD LSR II equipped with a UV laser. A singlet gate was established on the DAPI area vs. width scatter plot such that debris and doublets were excluded from the analysis, and at least 10,000 singlets were analyzed per sample. Secondary analysis was carried out using FCS express software. After doublet discrimination, a gating quadrant was applied to the scatter plot of side scatter versus CD3 fluorescence such that CD3 positive cells were positioned in the lower right quadrant. This gating quadrant was further refined by analysis of cytokeratin 8/18 (CK8/18) positive cells, positioning them in the upper right quadrant. This gating scheme was then applied directly to the scatter plot of side scatter versus Ki-67 fluorescence, such that high side scatter Ki-67 positive cells correspond to proliferating cells that are the same size and shape as tumor cells, and low side scatter Ki-67 positive cells correspond to proliferating cells that are the same size and shape as normal cells.

### Results

Analysis of cells dissociated from fixed tumors showed that they consisted of a mixture of tumor and normal cells. The cells dissociated from the same tumor consisted of a population of CD3 positive normal cells, and CK positive tumor cells (FIGS. 10A and 11A). Inspection of the DNA content of these two different marker positive populations revealed that CD3 positive cells contained diploid DNA content, while CK positive cells often contain aneuploid DNA content (FIG. 10B). Sometimes, the CK positive tumor cells were also diploid (FIG. 11B). When the cytokeratin positive tumor cell population was aneuploid, the high side scatter Ki-67-positive cells were also aneuploid, supporting that these were correctly identified as proliferating tumor cells (FIGS. 10C and 10D). When the cytokeratin positive population was diploid, the high side scatter Ki-67 positive cells showed a strong G2 DNA peak, consistent with a phenotype of proliferating cells (FIGS. 11C and 11D). The low side scatter Ki-67 positive cells were always diploid, suggesting these were proliferating normal cells (FIGS. 10C and 10D; and FIGS. 11C and 11D).

### Conclusion

Flow cytometric analysis of dissociated fixed tumor tissue was possible. The use of cytokeratin (CK) and CD3 to define the side scatter cutoff for tumor and normal cells assisted with gating of proliferating tumor and normal cells. This flow cytometric assay may be used to determine the percentage of proliferating tumor cells in the tumor microenvironment.

### Example 2

### Method

Same as described in Example 1.

### Results

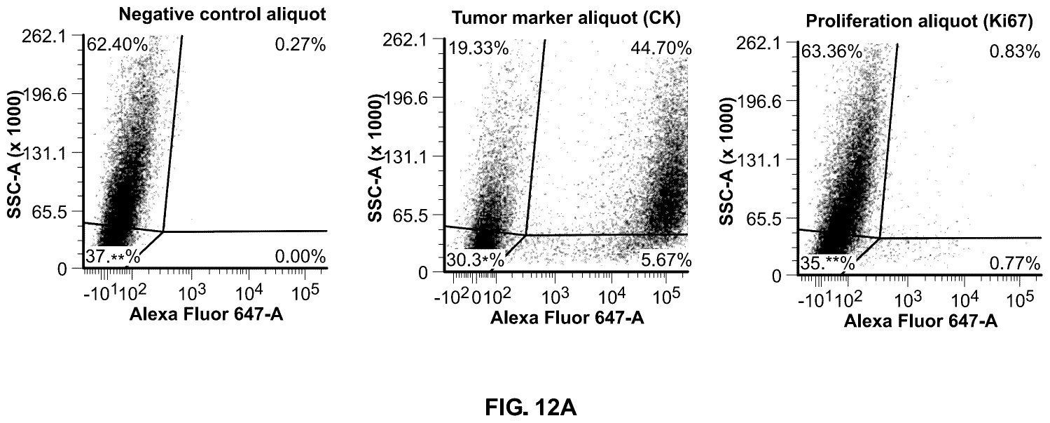

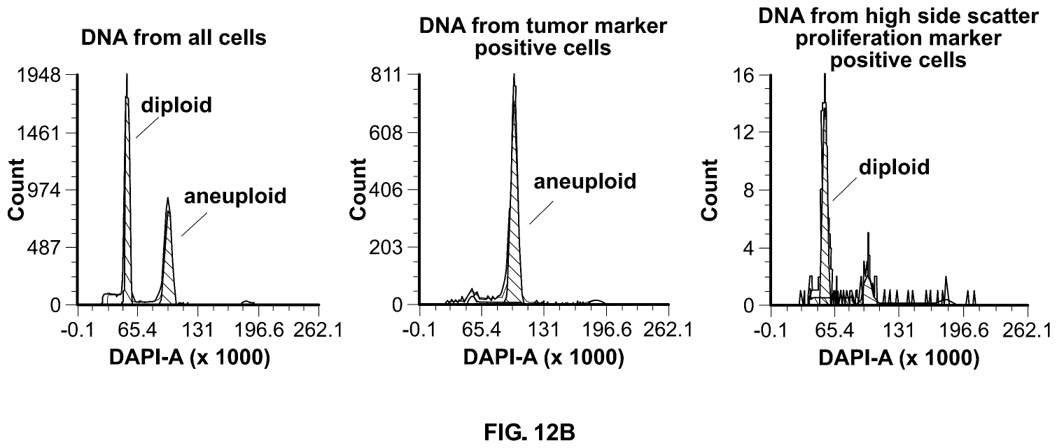

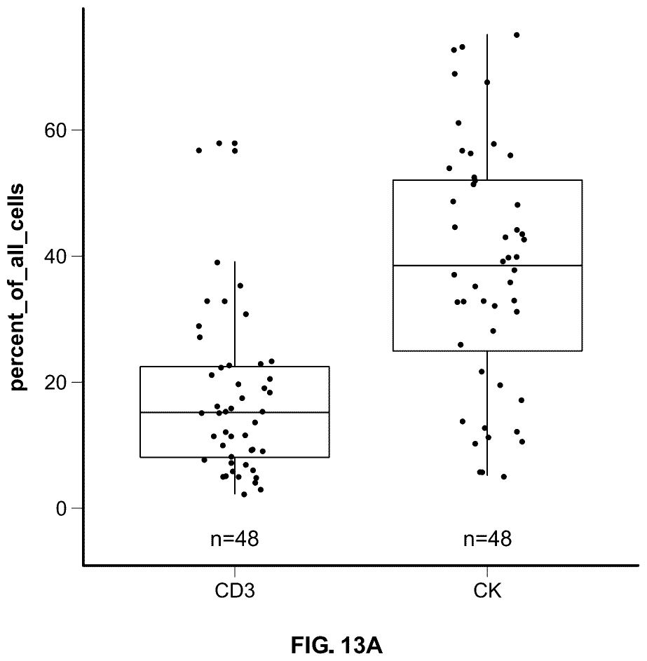

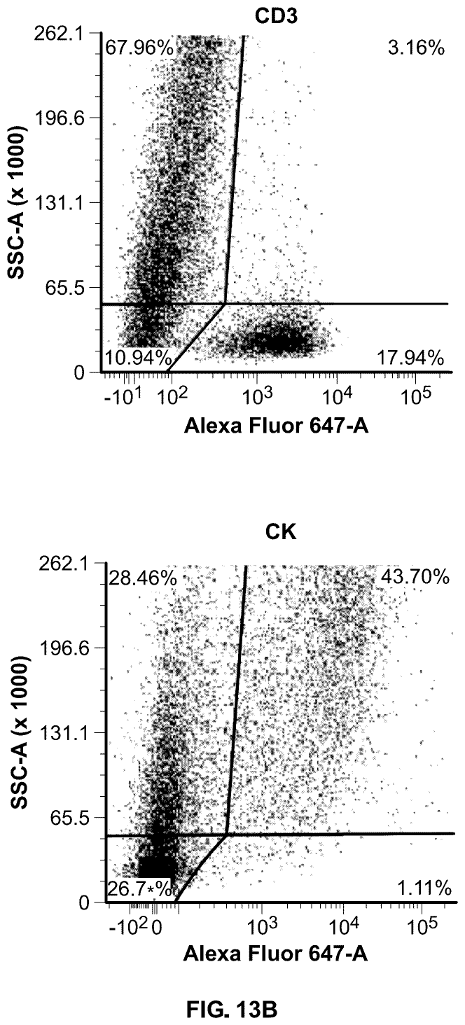

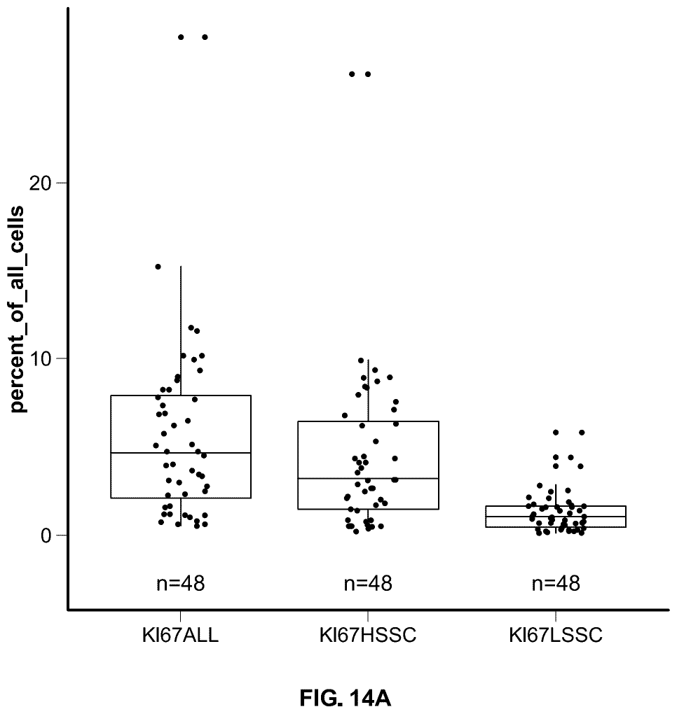

Analysis of an isolated case demonstrated a scenario in which DNA content of high side scatter Ki-67 positive cells can be used to exclude false positive results. The same gating principles were applied to this case as described in Example 1. A very low percentage of high side scatter Ki-67 positive cells (0.83%) was observed (FIG. 12A). Inspection of the DNA content of CK positive tumor cells revealed that the tumor cells are aneuploid (FIG. 12B, middle histogram), but the DNA content of the high side scatter Ki-67 positive cells is diploid (FIG. 12B, last histogram).

### Conclusion

In the previous example, which represented the vast majority of the cases analyzed, the DNA content of the high side scatter Ki-67 positive cells matched the DNA content of the CK positive tumor cells. The isolated case presented in this example represented either low-level non-specific Ki-67 staining, or low frequency normal proliferating cells that have high side scatter. The diploid DNA content of this low percentage population revealed that these were not tumor cells, and this case should not be considered to have proliferating tumor cells. Therefore, the data presented in this example in FIG. 12 demonstrated a rare case that should be excluded from analysis using this method.

### Example 3

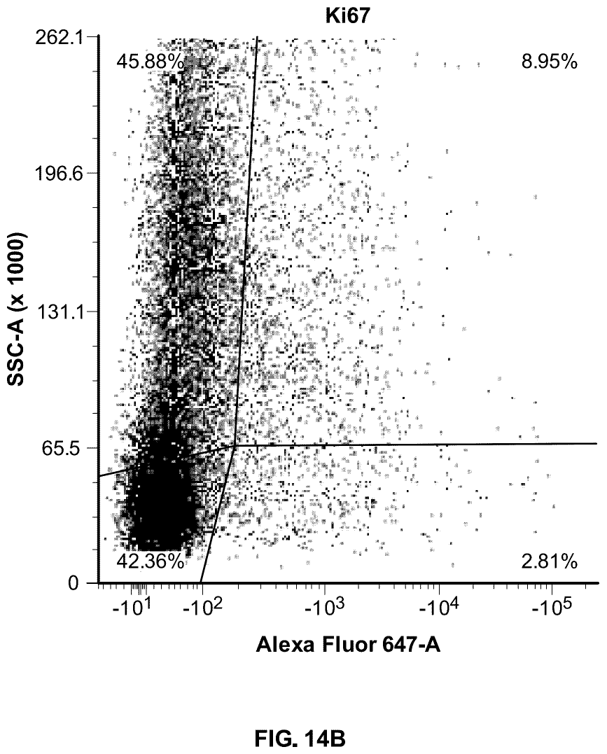

FIG. 14A represents a visualization of the diverse levels of Ki-67 positivity across 48 different breast cancer cases. FIG. 14B is a representative scatter plot to help define the populations displayed within the boxplots in FIG. 14A. “KI-67ALL” refers to the addition of both the upper right and lower right quadrants from the graph in FIG. 14B for each case. “KI-67 HSSC” refers to the percentage of positive cells in the upper right quadrant from the graph in FIG. 14B for each case. “KI-67 LSSC” refers to the percentage of positive cells in the lower right quadrant from the graph in FIG. 14B for each case.

As such, in FIG. 14A, the box plot labeled “KI-67ALL” shows the spread of the percentage of all proliferating cells in the tumor microenvironment across 48 different breast cancer patients. The box plot labeled “KI-67HSSC” represents the variation in proliferating tumor cells across 48 different breast cancer cases. The box plot labeled “KI-67LSSC” represents the variation in proliferating normal cells across the 48 breast cancer cases.

### Example 4—Insights From Residual Fixed Surgical Tissue in ER+ Breast Cancer

Introduction: In ER+ breast cancer, recent clinical trials (e.g., POETIC) explore the potential for pre-surgical treatment, and assessment of biomarker response in post-surgical tissue, to inform adjuvant treatment. In larger tumors, heterogeneous responses or subclonal mechanisms of resistance may be a challenge to assess with a single biopsy. Representative Sampling is a method wherein tumors dissected from residual formalin-fixed surgical tissue are homogenized, single cells isolated, biomarkers measured by flow cytometry, and specific cellular populations FACS sorted for genomic analysis. The goals of this ongoing study were to 1) assess the percentage of breast cancer cases with tissue available after FFPE sampling and 2) characterize genomic and phenotypic features of ER+ breast tumors using Representative Sampling methods (such as those described herein).

Methods: Patients were consented into the HoLST-F study (NCT03832062), and an interim analysis of cases with leftover tissue took place according to the study protocol. Tissues from 73 cases were dissected and homogenized. Cells from representative aliquots were analyzed on a BD LSRII flow cytometer for CK8/18, CD3, and Ki-67 using Ventana antibodies, and for tumor ploidy using DAPI. In a subset, CK8/18+ tumor cells were enriched using a BD FACS Melody sorter. Genomic DNA from enriched tumor cells and normal tissue was sequenced using an Agilent Exome panel, variants called using an in-house pipeline, and results assessed for the OncoKB level of clinical evidence.

Results: Interim feasibility analysis showed 25% of breast cancer cases had leftover tissue after FFPE sampling. Flow cytometric analysis of Ki-67 revealed a quantitative continuum of marker-positive tumor and normal cells across the cohort. Enriched tumor exome data identified clonal and subclonal clinically relevant variants. For one case, a low frequency ESR1 D538G mutation was identified, missed in the FFPE samples, which causes resistance to anti-endocrine treatment.

Conclusion: A complimentary workflow for fixed surgical tissue has value in breast cancer research and clinical studies. Ki-67 by flow cytometry from dissociated fixed tumors is a rapid quantitative method to assess proliferation, a feature that guides adjuvant treatment. Sequencing enriched tumor cells sampled from a larger portion of the tumor may improve sensitivity to detect low frequency variants missed by FFPE.

### Alternative Embodiments

In some embodiments, the disclosure provides a method of assessing a percentage of cell proliferation marker positive normal cells and a percentage of cell proliferation marker positive tumor cells comprising: obtaining at least two aliquots of a sample, wherein cells within a first aliquot of the at least two aliquots of the sample are stained for the presence of a cell proliferation marker, and wherein cells within a second aliquot of the at least two aliquots of the sample are stained for the presence of a tumor marker; generating a first scatter plot for the stained cells within the first aliquot of the sample; generating a second scatter plot for the stained cells within the second aliquot of the sample; and performing at least two sequential gating operations using at least the first and second generated scatter plots to assess the percentage of cell proliferation positive normal cells and the percentage of cell proliferation positive tumor cells.

In some embodiments, the disclosure provides a method of assessing a percentage of cell proliferation marker positive normal cells and a percentage of cell proliferation marker positive tumor cells comprising: obtaining a residual surgical tumor sample from a human subject, wherein the residual surgical tumor material is fixed but not embedded within paraffin, and wherein the residual surgical tumor material has not been deparaffinized; mechanically blending the obtained residual surgical tumor material to provide a representative sample, wherein any subpopulations of cells that were originally spatially segregated within the residual surgical tumor material are homogeneously distributed throughout the representative sample, and wherein any aliquot removed from the representative sample comprises one or more populations of subclones at a proportion at which they existed within the obtained residual surgical tumor sample; obtaining at least two aliquots of the representative sample, wherein cells within a first aliquot of the at least two aliquots of the representative sample are stained for the presence of a cell proliferation marker, and wherein cells within a second aliquot of the at least two aliquots of the representative sample are stained for the presence of a tumor marker; generating a first scatter for the stained cells within the first aliquot of the representative sample; generating a second scatter plot for the stained cells within the second aliquot of the representative sample; and performing at least two sequential gating operations using at least the first and second generated scatter plots to assess the percentage of cell proliferation positive normal cells and the percentage of cell proliferation positive tumor cells.

All the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A method of quantifying a percentage of normal cells and a percentage of tumor cells expressing a cell proliferation marker comprising:
obtaining a sample;
staining cells within a first aliquot derived from the sample for the presence of at least one cell proliferation marker;
staining cells within a second aliquot derived from the sample for the presence of at least one tumor marker;
optionally counterstaining the cells within the first and second aliquots for the presence of DNA;
generating scatter plots of fluorescence versus side scatter for the stained cells within each of the first and second aliquots;
based on the obtained scatter plots, gating the stained cells into at least one of a cell proliferation marker positive tumor cell population and a cell proliferation marker positive normal cell population; and
quantifying the percentage of the normal cells and the percentage of the tumor cells in each of the cell proliferation marker positive tumor cell and cell proliferation marker positive normal cell populations.

2. The method of claim 1, wherein the at least one tumor marker is a cytokeratin.

3. The method of claim 2, wherein the cytokeratin is CK8/18 or a pan-cytokeratin marker recognizing cytokeratins 1-8, 10, 14-16 and 19.

4. The method of claim 1, wherein the at least one cell proliferation biomarker is selected from the group consisting of Ki-67, Ki-S5, Ki-S2, p21, p27, Caspases, BAD, CD95, fas-ligand, and parp-proteins.

5. The method of claim 1, wherein the gating of the stained cells comprises performing at least two sequential gatings, wherein a first gating of the at least two sequential gatings comprises identifying cells that are positive for the tumor cell marker; and wherein a second gating of the at least two sequential gatings comprises mapping the first gating to the generated scatter plot corresponding to the first aliquot.

6. The method of claim 5, wherein the first gating comprises: (i) obtaining a scatter plot of fluorescence versus side scatter for a negative control aliquot derived from the sample; (ii) positioning a vertical quadrant gate such that fewer than a predetermined percentage of the stained cells in the negative control aliquot scatter plot are located to the right of the vertical quadrant gate; and (iii) positioning a horizontal quadrant gate in the generated scatter plot for the second aliquot such that fewer than a predetermined percentage of the stained cells in the generated scatter plot for the second aliquot are located in a lower right corner of the generated scatter plot of the second aliquot.

7. The method of claim 5, further comprising optionally assessing DNA content within at least the first and second aliquots to confirm the at least two sequential gatings.

8. The method of claim 1, wherein the obtained sample is derived from a heterogenous input sample which has been mechanically and/or chemically dissociated, and wherein the obtained sample comprises substantially uniformly distributed cells.

9. The method of claim 1, further comprising sequencing genomic material isolated from cells within the cell proliferation marker positive tumor cell population.

10. A method of assessing a percentage of cell proliferation marker positive normal cells and a percentage of cell proliferation marker positive tumor cells comprising:
(i) obtaining at least two aliquots of a sample, wherein cells within a first aliquot of the at least two aliquots of the sample are fluorescently stained for the presence of a cell proliferation marker, and wherein cells within a second aliquot of the at least two aliquots of the sample are fluorescently stained for the presence of a tumor marker;
(ii) generating a first scatter plot of fluorescence versus side scatter for the fluorescently stained cells within the first aliquot of the sample;
(iii) generating a second scatter plot of fluorescence versus side scatter for the fluorescently stained cells within the second aliquot of the sample; and
(iv) performing at least two gating operations using at least the first and second generated scatter plots to assess the percentage of cell proliferation positive normal cells and the percentage of cell proliferation positive tumor cells.

11. The method of claim 10, further comprising counterstaining cells within the first and second aliquots for the presence of DNA.

12. The method of claim 10, further comprising obtaining a third aliquot of the sample, wherein cells within the third aliquot of the sample are fluorescently stained for the presence of a normal cell marker.

13. The method of claim 10, wherein the tumor marker is selected from the group consisting of CK1, CK2, CK3, CK4, CK5, CK6, CK7, CK8, and CK9.

14. The method of claim 10, wherein the tumor marker is selected from the group consisting of CK10, CK12, CK13, CK14, CK16, CK17, CK18, CK19, and CK20.

15. The method of claim 10, wherein the tumor marker is CK8/18 or a pan-cytokeratin marker recognizing cytokeratins 1-8, 10, 14-16 and 19.

16. The method of claim 10, wherein the cell proliferation marker is selected from the group consisting of Ki-67, Ki-S5, Ki-S2, p21, p27, Caspases, BAD, CD95, fas-ligand, parp-proteins.

17. The method of claim 10, further comprising (i) obtaining a negative control aliquot, wherein cells within the negative control aliquot are incubated with one or more detection reagents; and wherein cytometry data is generated for the negative control aliquot; and (ii) generating a negative control scatter plot of fluorescence versus side scatter for the cells within the negative control aliquot.

18. The method of claim 10, wherein a first gating of the at least two gatings comprises identifying cells that are positive for the tumor cell marker; and wherein a second gating of the at least two gatings comprises identifying cell proliferation marker positive normal cells and cell proliferation marker positive tumor cells.

19. The method of claim 10, wherein the obtained sample is a representative sample derived from a heterogenous input sample which has been mechanically and/or chemically dissociated, and wherein the representative sample comprises substantially uniformly distributed cells, and wherein a ratio of cells in any aliquot derived from the representative sample is substantially similar to the ratio of cells in the heterogeneous input sample.

20. The method of claim 10, further comprising sorting the cell proliferation marker positive cells into a cell proliferation marker positive normal cell population and a cell proliferation marker positive tumor cell population; and sequencing genomic material isolated from cells within the cell proliferation marker positive tumor cell population.
